(19) United States
(12) Patent Application Publication
(10) Pub. No.: US 2025/0099205 A1
(43) Pub. Date: Mar. 27, 2025

(54) SYSTEM AND METHOD FOR ALIGNMENT AND REGISTRATION

(71) Applicant: Align Technology, Inc., San Jose, CA (US)

(72) Inventors: Guotu LI, Apex, NC (US); Yun GAO, Cary, NC (US); Christopher E. CRAMER, Durham, NC (US)

(21) Appl. No.: 18/898,623

(22) Filed: Sep. 26, 2024

Related U.S. Application Data
(60) Provisional application No. 63/585,581, filed on Sep. 26, 2023.

(51) Int. Cl. A61C 7/00; G06T 7/00; G06T 17/00

(52) U.S. Cl. CPC A61C 7/002; G06T 7/0012; G06T 17/00; G06T 2207/30036

(57) ABSTRACT

Apparatuses, systems, and methods for importing and/or transferring data, including dental data, from three-dimensional (3D) dental models to two-dimensional (2D) dental images may manipulate the 3D dental model based on a temporomandibular joint. A 2D projected image is generated from a patient's 3D dental model. If the 2D projected image matches (within a threshold) to a patient's 2D dental image (from a photo or the like), then dental data may be transferred. If the 2D projected image does not match, then the 3D dental model may be modified (based on the temporomandibular joint) and a new 2D projected image generated. In this manner, the positions of the 3D dental model may be iterated to generate multiple 2D projected images.

### CLAIM OF PRIORITY

This patent application claims priority to U.S. provisional patent application No. 63/585,581, titled “SYSTEM AND METHOD FOR ALIGNMENT AND REGISTRATION,” filed on Sep. 26, 2023 and herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The systems and methods described herein relate generally to dental models, and more particularly to the alignment and registration between different dental models associated with a patient.

### BACKGROUND

Orthodontic procedures typically involve repositioning a patient's teeth to a desired arrangement in order to correct malocclusions and/or improve aesthetics. To achieve these objectives, orthodontic appliances such as braces, shell aligners, and the like can be applied to the patient's teeth by an orthodontic practitioner and/or by the patients themselves. The appliance can be configured to exert force on one or more teeth in order to effect desired tooth movements according to a treatment plan.

Treatment planning, in general, may be used in any medical procedure to help guide a desired outcome. In some examples, orthodontic treatment planning may be used in orthodontic and dental treatments where a series of patient-removable appliances (e.g., orthodontic aligners, palatal expanders, and the like) are provided to correct a variety of different orthodontic or dental conditions. Thus, a treatment plan may be used to determine a number of intermediate stages (steps) as well as corresponding individual dental appliances (aligners) that are worn sequentially.

In some cases, a joint simulation of a patient's upper and lower jaws can provide a more comprehensive view of the patient's entire dentition which can improve determining a treatment plan.

### SUMMARY OF THE DISCLOSURE

Implementations address the need to provide a system for automatically, effectively, and accurately importing or capturing dental information (tooth numbering, and/or other dental features) from a three-dimensional (3D) scan, dataset, or model of a patient's detention to a two-dimensional (2D) dental image. The present application addresses these and other technical problems by providing technical solutions and/or automated agents that compare 3D scans, 3D datasets, or other 3D models to a 2D image. In some implementations, 3D models may be projected onto a plane to generate a 2D projection that may be compared to the 2D image. If the 2D projection matches (within a threshold) the 2D image, then dental information from the 3D models can be imported from (transferred to) the 2D image.

Described herein are apparatuses, systems, and methods for importing or transferring information from a 3D model to a 2D image. A 3D model may include a patient's lower jaw positioned with respect to the patient's upper jaw. The positioning may be based on a location of a temporomandibular joint that hinges the lower jaw to the upper jaw. In general, the 3D model may be positioned in a virtual 3D space and a virtual camera may be positioned with respect to the 3D model. A 2D projection of the 3D model is determined with respect to the virtual camera. The 2D projection is compared to a 2D dental image of the patient. If the 2D projection matches (within a threshold) the 2D dental image, then data from the 3D model can be imported to the 2D dental image.

Any of the methods described herein may include generating a three-dimensional (3D) alignment model based on a patient's 3D dental scans, wherein the 3D alignment model includes segmentation data of the patient's upper jaw portion and lower jaw portion, generating a two-dimensional (2D) alignment projection based on the 3D alignment model, determining a 2D differences between a patient's 2D dental image and the 2D alignment projection, and importing dental information from the patient's 3D dental scan to the patient's 2D dental image when the difference is less than a threshold.

In any of the methods, generating the 3D alignment model may include determining a position of the lower jaw portion with respect to the upper jaw portion. In some examples, the position of the lower jaw portion may be constrained by a location of a joint coupling the lower jaw portion to the upper jaw portion. Furthermore, in some examples the position of the lower jaw portion may be determined, at least in part, by a temporomandibular joint disposed with respect to the upper jaw portion.

In any of the methods described herein, generating the 3D alignment model may include moving the lower jaw portion with respect to the upper jaw portion. In some examples, moving the lower jaw portion is based on the location of a joint coupling the lower jaw portion to the upper jaw portion.

In general, generating the 2D alignment projection may include determining the location of the 3D alignment model and a location of a projection plane in a common virtual 3D space. In some examples, generating the 2D alignment projection may include projecting dental elements from the 3D alignment model to the projection plane, wherein the 2D alignment projection is based on the projected dental elements. In some other examples, the location of the 3D alignment model and the location of the projection plane is based, at least in part, on a point-of-view associated with a virtual camera disposed in the common virtual 3D space.

In some variations, generating the 2D alignment projection may include cropping a portion of the projection plane prior to determining 2D differences between the patient's 2D dental image and the 2D alignment.

In any of the methods described herein, determining the location of the 3D alignment model includes locating the patient's upper jaw portion in 3D space using six degrees of freedom and locating the patient's lower jaw portion in the 3D space using one degree of freedom.

In any of the methods described herein, generating the 2D alignment projection may include iteratively determining a position of the projection plane based on the difference between the patient's 2D dental image and the 2D alignment projection.

Generally, in any of the methods described herein, the 2D dental image may be based on a photo of the patient's dentition. In some variations, the 2D dental image may include 2D information of an upper jaw portion and a lower jaw portion.

In any of the methods described herein, determining the 2D differences between the patient's 2D dental image and the 2D alignment projection may include determining a difference between corresponding features of the patient's 2D dental image and the 2D alignment projection.

In some variations, determining the 2D differences between the patient's 2D dental image and the 2D alignment projection may include determining a difference between outlines of corresponding dental structures of the patient's 2D dental image and the 2D alignment projection.

In still other variations, determining the difference between the patient's 2D dental image and the 2D alignment projection may include determining a difference between tooth boundaries determined from the patient's 2D dental image and the 2D alignment projection.

In any of the methods described herein, the segmentation data may be generated with one or more machine learning engines and one or more 3D models. Furthermore, in any of the methods described herein, the dental information may include tooth number information. Generally, the 2D dental image is based on a photo of the patient's dentition.

In any of the methods, generating the 3D alignment model may further comprise iteratively determining a position of the lower jaw portion with respect to the upper jaw portion based on the difference between the patient's 2D dental image and the 2D alignment projection.

In some examples, generating the 2D alignment projection may include iteratively determining a position of a projection plane based on the difference between the patient's 2D dental image and the 2D alignment projection.

In general in any of the methods described herein may further include generating a 3D image based on the 2D alignment projection. In some variations, in any of the methods described herein the patient's 2D dental image is a closed-bite photo. In some examples, the patient's 3D dental scans may be associated with a previously determined treatment plan.

For example, any of these methods may include importing dental information from the patient's 3D dental scan and may include determining a bite class from the 3D dental scan. For example, determining the bite class from the 3D dental scan may comprise identifying one or more of: class I malocclusion, class II malocclusion, class III malocclusion, crossbite, deep bite, and/or open bite. Any of these methods may include measuring a degree of malocclusion from the patient's 3D dental scan.

Any of these methods may include determining or correcting tooth numbering using the patient's 3D dental scan.

For example, a method may include: generating or accessing a three-dimensional (3D) alignment model of a digital model of a patient's upper jaw portion and a digital model of the patient's lower jaw portion, wherein the digital model of the patient's upper jaw portion and the digital model of the patient's lower jaw portion are based on one or more intraoral scans, wherein the 3D alignment model includes a TMJ parameter; generating a two-dimensional (2D) alignment projection image from the 3D alignment model; determining a difference estimate between a 2D dental image of the patient's teeth and the 2D alignment projection; iteratively adjusting the TMJ parameter based on the difference estimate and repeating the steps of generating the 2D alignment projection image and determining the difference estimate until the difference is less than a threshold or until the number of iterations exceeds a second threshold; and outputting the 3D alignment model including the TMJ parameter.

Also described herein are systems configured to perform any of these method. For example, a system comprising: one or more processors; and a memory configured to store instructions that, when executed by the one or more processors, cause the system to: generate a three-dimensional (3D) alignment model based on a patient's 3D dental scans, wherein the 3D alignment model includes segmentation data of the patient's upper and lower jaw; generate a two-dimensional (2D) alignment projection based on the 3D alignment model; determine a 2D difference between a patient's 2D dental image and the 2D alignment projection; and import dental information from the patient's 3D dental scan to the patient's 2D dental image when the difference is less than a threshold.

For example, described herein are non-transitory computer-readable storage medium comprising instructions that, when executed by one or more processors of a device, cause the device to perform operations comprising: generating a three-dimensional (3D) alignment model based on a patient's 3D dental scans, wherein the 3D alignment model includes segmentation data of the patient's upper and lower jaw; generating a two-dimensional (2D) alignment projection based on the 3D alignment model; determining a 2D difference between a patient's 2D dental image and the 2D alignment projection; and importing dental information from the patient's 3D dental scan to the patient's 2D dental image when the difference is less than a threshold.

As mentioned, any of these methods may be configured as a method of determining a bite class of a patient. These methods may include: accessing or receiving one or more 2D images of a patient's dentition; registering a 3D digital model of a patient's dentition using the one or more 2D images so that a 3D digital model of an upper jaw of the patient and a 3D digital model of a lower jaw of the patient are registered to each other so that the 3D digital model may provide a relative movement of the 3D digital model of the lower jaw relative to the 3D digital model of the upper jaw; determining the bite class of the patient from the registered 3D digital model; and outputting the bite class.

The bite class may be one or more of: class I malocclusion, class II malocclusion, class III malocclusion, crossbite, deep bite, and/or open bite. For example, determining the bite class may comprise measure the degree of malocclusion from the 3D digital model. In any of these methods outputting the bite class may comprise displaying the bite class on a user interface.

Any of the systems described herein may include one or more processors and a memory that is configured to store instructions that, when executed by the one or more processors cause the system to generate a three-dimensional (3D) alignment model based on a patient's 3D dental scans, wherein the 3D alignment model includes segmentation data of the patient's upper and lower jaw, generate a two-dimensional (2D) alignment projection based on the 3D alignment model, determine a 2D differences between a patient's 2D dental image and the 2D alignment projection, and import dental information from the patient's 3D dental scan to the patient's 2D dental image when the difference is less than a threshold.

Any of the non-transitory computer-readable storage mediums described herein may include instructions that, when executed by one or more processors of a device, cause the device to perform operations comprising generating a three-dimensional (3D) alignment model based on a patient's 3D dental scans, wherein the 3D alignment model includes segmentation data of the patient's upper and lower jaw, generating a two-dimensional (2D) alignment projection based on the 3D alignment model, determining a 2D differences between a patient's 2D dental image and the 2D alignment projection, and importing dental information from the patient's 3D dental scan to the patient's 2D dental image when the difference is less than a threshold.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A is a diagram showing an example of a computing environment configured to facilitate gathering and processing digital scans of a dental arch with teeth and/or bones therein.

FIG. 1B is a diagram showing an example of the scan segmentation engine(s).

FIG. 1C shows a diagram that includes an example of the 3D fusion engine(s).

FIG. 1D shows a diagram that includes an example of the tooth modeling engine(s).

FIG. 1E shows a diagram that includes an example of the tooth labeling engine(s).

FIG. 1F shows a diagram that includes an example of the 2D alignment engine.

FIG. 2 shows an example 3D representation of a patient's dentition.

FIG. 3 shows an example virtual 3D space.

FIG. 4 is a flowchart showing an example method for determining a match between a 2D projection and a 2D dental image.

FIGS. 5A-5C show images associated with an open bite, as described herein.

FIGS. 6A-6C show images associated with a closed bite, as described herein.

FIGS. 7A-7C show images associated with determining tooth numbers based on 2D dental images.

FIG. 8 is a block diagram of a device that may be one example of a device configured to perform one or more operations described herein.

FIG. 9 is a diagram of one example of a computing environment within which the methods and apparatuses described herein may be implemented.

FIG. 10 is a schematic illustration of one example of a treatment monitoring module as described herein.

### DETAILED DESCRIPTION

In general, the methods and apparatuses described herein may include simulating and/or modeling both a patient's upper and lower jaws together. In some examples the upper and lower jaws may be integrated into a single model and/or may include a description of the patient's temporomandibular joint (TMJ). The description of the TMJ may provide a relationship between the upper and lower jaws. In some cases the upper jaw and lower jaw may be separately modeled, but may be related by the description the patient's TMJ. In some cases the upper and lower jaw and TMJ may be part of a single model. The model may be a digital model. Any appropriate digital model format may be used, including point cloud, mesh, etc. In practice, these methods and apparatuses may be useful for modeling and visualization, including visualizing, displaying and modifying the patient's jaws, and for treatment planning, and/or designing (including simulating and testing) one or more dental appliances, and the like. As one non-limiting example, these methods and apparatuses may be useful for monitoring of anterior deep bite correction or any other condition, particularly those involving the engagement between the upper and lower jaws. These methods and apparatuses for implementing or using them, in which both the upper and lower jaw are modeled together, as well as the TMJ relationship may provide a significant advance as compared to techniques that do separate upper jaw and lower jaw registration, particularly in situations in which a portion of the upper or lower jaw is occluded, which may prevent accurate modeling of the relationship between the upper and lower jaws.

The apparatuses and/or methods described herein may be useful in planning and fabrication of dental appliances, including elastic polymeric positioning appliances, is described in detail in U.S. Pat. No. 5,975,893, and in published PCT application publication No. WO 98/58596, which is herein incorporated by reference for all purposes. Systems of dental appliances employing technology described in U.S. Pat. No. 5,975,893 are commercially available from Align Technology, Inc., San Jose, Calif., under the tradename, Invisalign System.

Throughout the body of the Description of Embodiments, the use of the terms “orthodontic aligner”, “aligner”, or “dental aligner” is synonymous with the use of the terms “appliance” and “dental appliance” in terms of dental applications. For purposes of clarity, embodiments are hereinafter described within the context of the use and application of appliances, and more specifically “dental appliances.”

A “patient,” as used herein, may be any subject (e.g., human, non-human, adult, child, etc.) and may be alternatively and equivalently referred to herein as a “patient” or a “subject.” A “patient,” as used herein, may but need not be a medical patient. A “patient,” as used herein, may include a person who receives orthodontic treatment, including orthodontic treatment with a series of orthodontic aligners.

In any of these methods and apparatuses, the method may include registration, e.g., alignment, of the three-dimensional (3D) model to the two-dimensional (2D) image, including a projection of a 3D model onto a 2D model. For example, these methods ma include registration (3D to 2D) using one or more camera parameters (e.g., location, rotation, etc.) with respect to a both jaws; thus, the methods described herein may coordinate three objects: the upper jaw (e.g., 3D model), the lower jaw (e.g., 3D model) and the camera. This coordination may provide the relationship between the upper and lower jaws, and may allow for accurate and rapid assessment of the patient's bite using both jaws. In contrast to currently used methods, separate modeling of the lower and upper jaws may be much less accurate (particularly where lower jaw information is missing or lacking), and may take significantly longer. Thus, in general, these methods may use joint jaw-pair optimization, determining the TMJ relationship between the patient's upper and lower jaw.

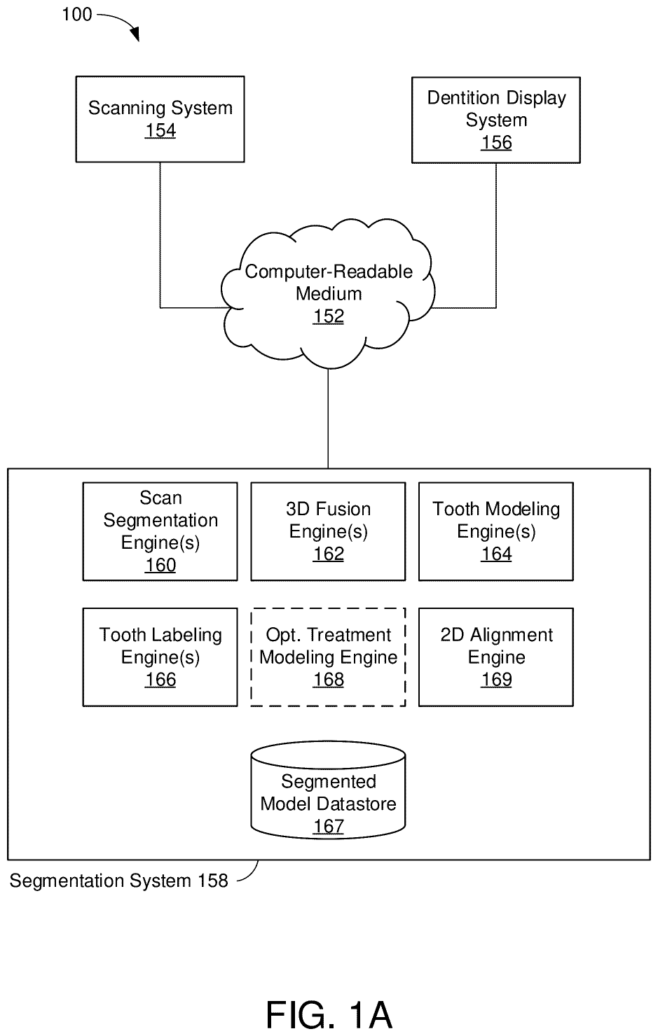

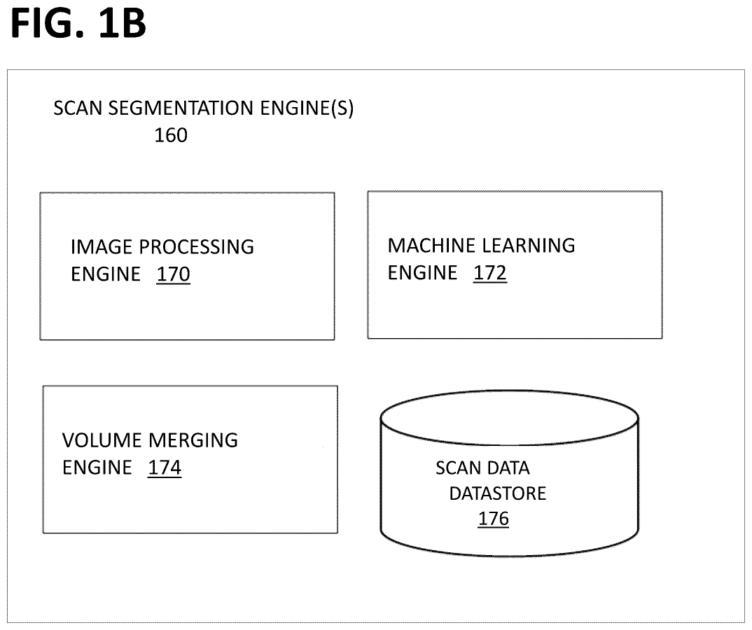

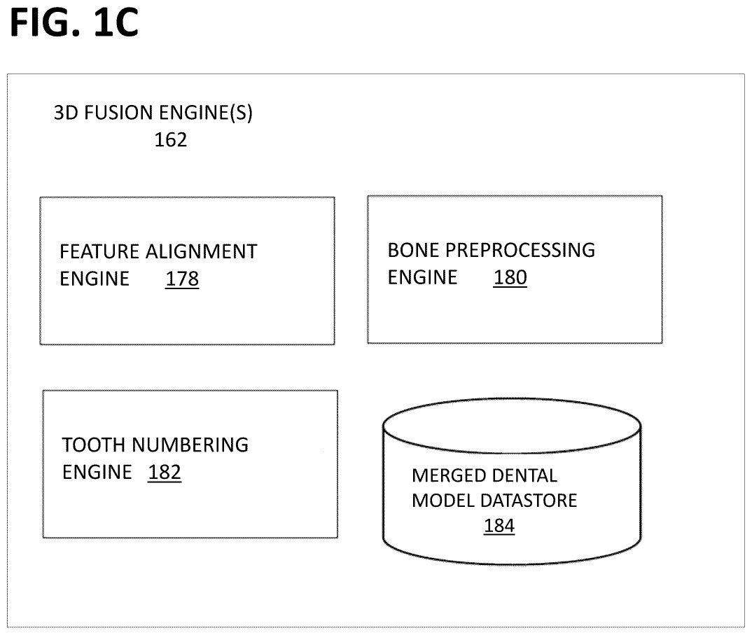

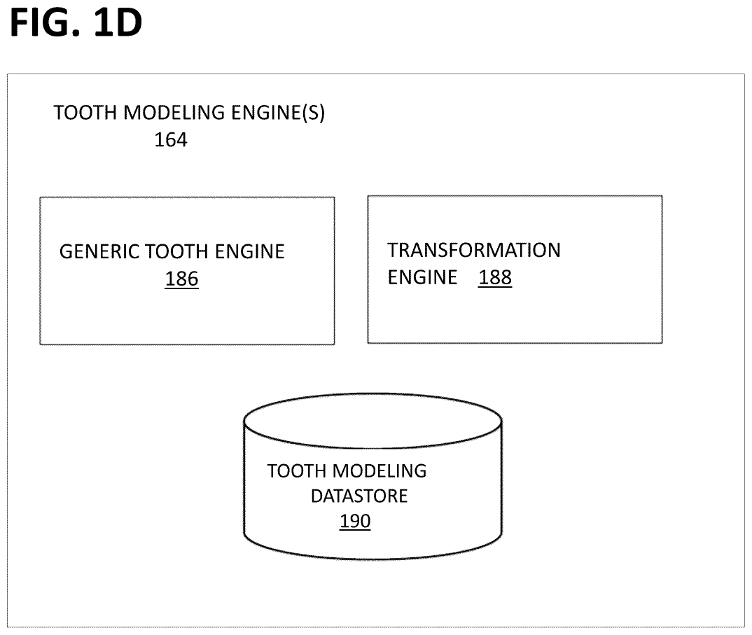

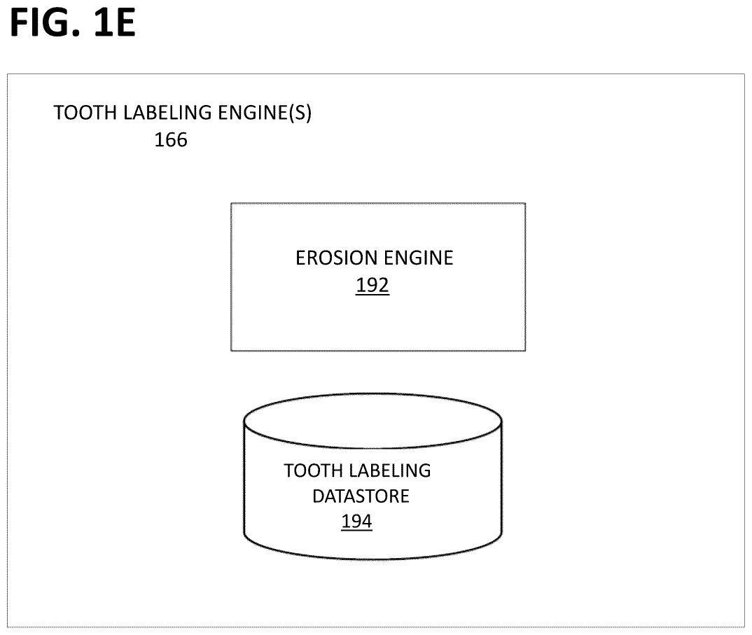

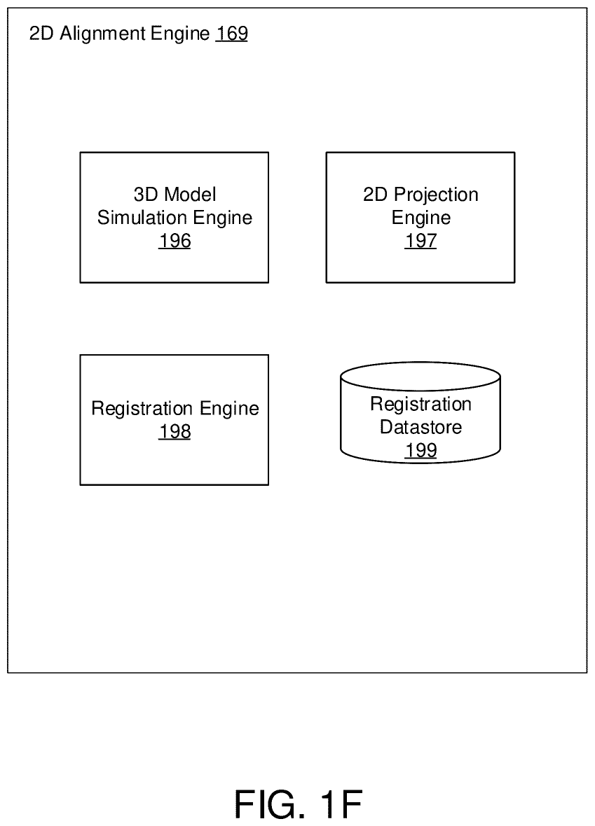

For example, FIG. 1A is a diagram showing an example of a computing environment 100 configured to facilitate gathering and processing digital scans of a dental arch with teeth and/or bones therein. The environment 100 includes a computer-readable medium 152, a scanning system 154, a dentition display system 156, and a segmentation system 158. One or more of the modules in the computing environment 100 may be coupled to one another or to modules not explicitly shown.

The computer-readable medium 152 and other computer readable media discussed herein are intended to represent a variety of potentially applicable technologies. For example, the computer-readable medium 152 can be used to form a network or part of a network. Where two components are co-located on a device, the computer-readable medium 152 can include a bus or other data conduit or plane. Where a first component is co-located on one device and a second component is located on a different device, the computer-readable medium 152 can include a wireless or wired back-end network or LAN. The computer-readable medium 152 can also encompass a relevant portion of a WAN or other network, if applicable.

The scanning system 154 may include a computer system configured to scan a patient's dental arch. A “dental arch,” as used herein, may include at least a portion of a patient's dentition formed by the patient's maxillary and/or mandibular teeth, when viewed from an occlusal perspective. A dental arch may include one or more maxillary or mandibular teeth of a patient, such as all teeth on the maxilla or mandible or a patient. The scanning system 154 may include memory, one or more processors, and/or sensors to detect contours on a patient's dental arch. The scanning system 154 may be implemented as a camera, an intraoral scanner, an x-ray device, an infrared device, a medical scanning device (e.g., CT scanner, CBCT scanner, MRI scanner) etc. In some implementations, the scanning system 154 is configured to produce three-dimensional (3D) scans of the patient's dentition. In other implementations the scanning system 154 is configured to produce two-dimensional (2D) scans or images of the patient's dentition. The scanning system 154 may include a system configured to provide a virtual representation of a physical mold of patient's dental arch. The scanning system 154 may be used as part of an orthodontic treatment plan. In some implementations, the scanning system 154 is configured to capture a patient's dental arch at a beginning stage, an intermediate stage, etc. of an orthodontic treatment plan. The scanning system 154 may be further configured to receive 2D or 3D scan data taken previously or by another system.

The dentition display system 156 may include a computer system configured to display at least a portion of a dentition of a patient. The dentition display system 156 may include memory, one or more processors, and a display device to display the patient's dentition. The dentition display system 156 may be implemented as part of a computer system, a display of a dedicated intraoral scanner, etc. In some implementations, the dentition display system 156 facilitates display of a patient's dentition using scans that are taken at an earlier date and/or at a remote location. It is noted the dentition display system 156 may facilitate display of scans taken contemporaneously and/or locally to it as well. As noted herein, the dentition display system 156 may be configured to display the intended or actual results of an orthodontic treatment plan applied to a dental arch scanned by the scanning system 154. The results may include 3D virtual representations of the dental arch, 2D images or renditions of the dental arch, etc.

The segmentation system 158 may include a computer system, including memory and one or more processors, configured to process scan data from the scanning system 154. In some examples, the 2D or 3D scan data can be segmented into individual dental components and processed into a 3D model of the patient's teeth. The 3D segmentation system can be configured to input one or more different areas of the 2D scan, 3D scan, or 3D model into a machine learning model to automatically segment the scan or model into individual dental components, including segmenting the scan or model into individual teeth, bones, interproximal spaces between teeth, and/or gingiva. The segmented 2D/3D scan or model can be used to create and implement a dental treatment plan for the patient. For example, a digital treatment planning software may incorporate the segmentation system 158 and receive a 3D scan of the patient's dentition. The segmentation system 158 may then be configured to automatically segment the 3D scan. The digital treatment planning software may then be configured to automatically generate a dental treatment plan for the patient, which may further include generating a 3D model of the patient's dentition that includes the 3D segmentation. The segmentation system 158 may include scan segmentation engine(s) 160, 3D fusion engine(s) 162, tooth modeling engine(s) 164, tooth labeling engine(s) 166, an optional treatment modeling engine(s) 168, a 2D alignment engine 169, and a segmented model datastore 167. One or more of the modules of the segmentation system 158 may be coupled to each other or to other modules not shown.

The scan segmentation engine(s) 160 of the segmentation system 158 may implement automated agents to process 2D or 3D scans taken by the scanning system 154. In some implementations, the scan segmentation engine(s) 160 formats scan data from a scan of a dental arch into one or more partitions, volumes, crops, or areas of the scan. The scan segmentation engine(s) 160 may be integrated into a digital treatment planning software. The one or more partitions, volumes, crops, or areas of the scan can be a subset or section of the original scan. In some implementations, the one or more partitions, volumes, crops, or areas of the scan can have a resolution different than the resolution of the original 2D or 3D scan. For example, the one or more partitions, volumes, crops, or areas of the scan can have a lower resolution than the original scan. In other implementations, the one or more partitions, volumes, crops, or areas of the scan can have the same resolution of the original scan. The scan segmentation engine(s) 160 can be further configured to implement automated agents to segment the 2D or 3D scan. In one implementation, the scan segmentation engine(s) can input the or more partitions, volumes crops, or areas of the scan into one or more machine learning models for segmentation into individual dental features such as upper jaw, lower jaw, and binary teeth segmentation. The segmentations of the one or more partitions, volumes, crops, or areas of the scan can be merged to generate full semantic segmentation of the 2D or 3D scan.

The 3D fusion engine(s) 162 of the segmentation system 158 can implement automated agents to align segmented scan data from the scan segmentation engine(s) 160 with a digital dental 3D treatment plan of the patient. The 3D fusion engine(s) 162 may be integrated into a digital treatment planning software. In some implementations, the 3D fusion engine(s) 162 provides coarse alignment of segmented scan data and triangulation of each labeled volume with corresponding dental features of the digital dental 3D treatment plan. The 3D fusion engine(s) 162 can then provide fine alignment of the segmented scan data with the dental treatment plan. In some implementations, the 3D fusion engine(s) 162 can preprocess the aligned segmented scan data and digital treatment plan for reduction of digital noise and suppression of potential segmentation errors. The 3D fusion engine(s) 162 can be further configured to accurately number individual teeth in the digital treatment plan. Additionally, the 3D fusion engine can implement automated agents to stitch scan data representing tooth crowns to digital treatment plan data representing tooth roots, providing the best possible resolution in the final segmented digital treatment plan.

The tooth modeling engine(s) 164 may implement automated agents to replace or modify low-quality or low-resolution segmentation data from the 2D/3D scan with higher quality generic tooth models. The tooth modeling engine(s) 164 may be integrated into a digital treatment planning software. In one implementation, the tooth modeling engine(s) 164 may be configured to identify a segmented tooth from the segmented scan data and identify a generic tooth model corresponding to the segmented tooth. In one implementation, the tooth modeling engine(s) 164 may implement automated agents to fit the generic tooth model into the segmented tooth. The generic tooth model can be modified/adjusted/rotated to precisely fit within the segmented tooth. The tooth modeling engine(s) 164 may then be configured to implement automated agents to transform the adjusted generic tooth model into the digital treatment plan for the patient. This process can be repeated for all the segmented teeth from the 2D/3D scan.

The tooth labeling engine(s) 166 may implement automated agents to label segmented teeth of segmented 2D/3D scan from the scan segmentation engine(s) 160. The tooth labeling engine(s) may be integrated into a digital treatment planning software. In one implementation, the tooth labeling engine(s) 166 receives the 2D/3D segmented scan. The tooth labeling engine(s) 166 can apply a morphological erosion algorithm to the segmented scan to divide the segmented scan into N voxel volumes, where N is the number of teeth in the segmented scan.

The optional (as denoted in FIG. 1A by dashed lines) treatment modeling engine(s) 168 may be configured to use the segmented 3D model, segmented scan, and/or the combination of the segmented scan and the digital dental model to store and/or provide instructions to implement orthodontic treatment plans and/or the results of orthodontic treatment plans. The treatment modeling engine(s) 168 may be integrated into a digital treatment planning software. The optional treatment modeling engine(s) 168 may provide the results of orthodontic treatment plans on a 3D model. In some embodiments, the 3D model can be rendered into one or more 2D image(s) from a plurality of viewing angles. The optional treatment modeling engine(s) 168 may model the results of application of orthodontic aligners to the patient's dental arch over the course of an orthodontic treatment plan. In some embodiments, the treatment modeling engine(s) 168 can be configured to save, transmit, or output the digital dental model and/or a digital orthodontic treatment plan. In some embodiments, the digital dental model and/or a digital orthodontic treatment plan can be displayed on a display for a user of the digital treatment planning software, such as a physician. The user can edit or make changes to the proposed digital dental model and/or a digital orthodontic treatment plan, such as by interacting with a user input device (e.g., mouse and keyboard, touchscreen, joystick, etc.) of the digital treatment planning software.

The 2D alignment engine 169 may determine an alignment between a segmented 3D model and a 2D dental image. The 2D alignment engine 169 may be integrated into a digital treatment planning software. The segmented 3D model, which may be determined by one or more modules of the segmentation system 158 may include upper jaw and lower jaw portions. In some implementations, the upper jaw portion may be independent of the lower jaw portion. However, since the lower jaw portion is physically constrained to the upper jaw portion through a patient's temporomandibular joint, the lower jaw portion may be limited in possible positions with respect to a 3D model of a patient's dentition that includes both the upper and lower jaw portions. The 2D alignment engine 169 can determine an alignment between a patient's 3D model and a 2D image of the patient's dentition. After determining the alignment between the 3D model and the 2D image, information from the patient's 3D model can be used to identify one or more portions or elements included in the 2D image. For example, teeth numbering information from the 3D model can be used to number teeth in the 2D image. The 2D alignment engine 169 is described in more detail below in conjunction with FIGS. 1F-8.

The segmented model datastore 167 stores segmented images including a patient's segmented 3D models generated by one or more of the modules within the segmentation system 158. For example, the segmented model datastore 167 may include a patient's segmented 3D models that include tooth numbering information. The segmented model datastore 167 may also include a patient's 2D images that have been aligned with one or more 3D models and can include tooth numbering information.

As used herein, any “engine” may include one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors or a first engine and a second engine can share one or more processors with one another or other engines. Depending upon implementation-specific or other considerations, an engine can be centralized, or its functionality distributed. An engine can include hardware, firmware, or software embodied in a computer-readable medium for execution by the processor. The processor transforms data into new data using implemented data structures and methods, such as is described with reference to the figures herein. In some examples, the engines discussed herein can be implemented in a digital orthodontic treatment planning software.

The engines described herein, or the engines through which the systems and devices described herein can be implemented as, for example, cloud-based engines. As used herein, a cloud-based engine is an engine that can run applications and/or functionalities using a cloud-based computing system. All or portions of the applications and/or functionalities can be distributed across multiple computing devices, and need not be restricted to only one computing device. In some embodiments, the cloud-based engines can execute functionalities and/or modules that end users access through a web browser or container application without having the functionalities and/or modules installed locally on the end-users' computing devices.

As used herein, “datastores” may include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

Datastores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores described herein can be cloud-based datastores. A cloud-based datastore is a datastore that is compatible with cloud-based computing systems and engines.

FIG. 1B is a diagram showing an example of the scan segmentation engine(s) 160. The scan segmentation engine(s) 160 may include an image processing engine 170, a machine learning engine 172, a volume merging engine 174, and a scan data datastore 176. One or more of the modules of the scan segmentation engine(s) 160 may be coupled to each other or to modules not shown.

The image processing engine 170 may implement one or more automated agents configured to format 2D or 3D scan data from a scan of a dental arch scan data into one or more partitions, volumes, crops, or areas of the scan. For example, the image processing engine may receive or access a 3D scan of a patient's dentition, such as a CT scan, a CBCT scan, or an MRI scan, which can include high-resolution imaging data of the patient's dental features, including the patient's teeth and the upper and lower jaw bones of the patient's jaw. The image processing engine can then process the scan into one or more partitions, volumes, crops, or areas of the scan which may be a subset of the original scan. For example, the one or more partitions, volumes, crops, or areas of the scan can be, for example, a crop with data only representing the upper bone of the jaw, the lower bone of the jaw, and/or the teeth of the patient. In one implementation, the image processing engine can take into account specific geometric features of the 2D/3D scan to determine how/where to crop the scan. For example, the image processing engine can implement a center of teeth area computation to determine where the patient's teeth are located in the 2D/3D scan.

The image processing engine 170 can calculate a center of teeth areas estimation, which can then be used by the system to generate one or more partitions, volumes, crops, or areas of the scan that include scan data of the patient's teeth. The image processing engine 170 may be further configured to implement automated agents to resample or adjust the resolution of the 2D/3D scan or of the one or more partitions, volumes, crops, or areas of the scan. For example, in one implementation, the entire 2D/3D scan may be resampled to have a lower resolution than the native resolution of the scan. In another implementation, one or more partitions, volumes, crops, or areas of the scan may be resampled to have a different (e.g., lower) resolution. The image processing engine 170 may provide the processed scan data and/or other data to the scan data datastore 176.

The machine learning engine 172 may implement one or more automated agents configured to apply one or more machine learning engines to segment the processed scan data from the image processing engine. For example, the machine learning engine 172 can use, as an input, the original 2D/3D scan (e.g., a CT scan, CBCT scan, or MRI scan) and/or the one or more partitions, volumes, crops, or areas of the scan from the image processing engine. As described above, the one or more partitions, volumes, crops, or areas of the scan may also have various resolutions, as some of the crops may have a lower resolution than the original 2D/3D scan. A plurality of the aforementioned inputs may be used to generate segmentation data. For example, a low-resolution version of the 2D/3D scan may be input into the machine learning engine to generate an upper jaw/lower jaw/binary teeth segmentation. Additionally, one or more partitions, volumes, crops, or areas of the scan at different resolutions can be input into the machine learning engine to generate segmentation data. Higher resolution crops of the patient's teeth can be input into the machine learning engine to generate segmentation data of the patient's teeth. Additionally, lower resolution crops of the patient's upper/lower jaw can be input into the machine learning engine to generate segmentation data. The machine learning engine 172 may provide the segmented data and/or other data to the scan data datastore 176.

Examples of machine learning systems that may be used by the machine learning engine include, but are not limited to, Convolutional Neural Networks (CNN) such as V-net, U-Net, ResNeXt, Xception, RefineNet, Kd-Net, SO Net, Point Net, or Point CNN, and additional machine learning systems such as Decision Tree, Random Forest, Logistic Regression, Support Vector Machine, AdaBoosT, K-Nearest Neighbor (KNN), Quadratic Discriminant Analysis, Neural Network, etc. Additionally, the variations of the CNNs described above can be implanted. For example, a CNN such as Unet can be modified to use alternative convolutional blocks (e.g., ResNeXt or Xception) instead of the VGG-style blocks that are implemented by default.

The volume merging engine 174 may implement one or more automated agents configured to merge the segmented data from the machine learning engine 172 into a full semantic segmentation of the 2D or 3D scan, including segmentation of the patient's upper jaw/lower jaw/individual teeth. As described above, the machine learning engine may provide segmentation data from various scan data inputs, including segmenting the original 2D/3D scan, segmenting a resampled (e.g., low resolution) scan, and/or segmenting one or more partitions, volumes, crops, or areas of the scan. The resulting segmentation data comprises a plurality of segmented volumes, each volume potentially having varying resolutions and pertaining to varying locations within the original 2D/3D scan. The volume merging engine 174 can be configured to implement automated agents to merge the segmented volumes from the machine learning engine into a single, comprehensive segmentation of the original 2D or 3D scan. For example, the volume merging engine 174 can be configured to merge a first volume (e.g., a low-resolution upper jaw/lower jaw/binary teeth segmentation volume) and a second volume (e.g., a high-resolution, multi-class teeth segmentation volume) using the following steps: 1) remove binary teeth labels from the first volume; 2) adjust the resolution of the first volume to be the same as the second volume; and 3) replace voxels in the first volume with voxels from the second volume. The resulting volume contains information about high-resolution teeth and low-resolution upper jaw and lower jaw areas.

The scan data datastore 176 may be configured to store data related to the 2D or 3D scan, the cropped or resampled scan data, the segmented scan data, and/or the merged volume data from the modules described above.

FIG. 1C shows a diagram that includes an example of the 3D fusion engine(s) 162. The 3D fusion engine(s) 162 may include a feature alignment engine 178, a bone preprocessing engine 180, a tooth numbering engine 182, and a merged dental model datastore 184. One or more of the modules of the 3D fusion engine(s) 162 may be coupled to each other or to modules not shown.

The feature alignment engine 178 may implement one or more automated agents configured to align and merge segmented scan data from the scan segmentation engine(s) 160 with a digital 3D dental treatment plan. A digital 3D dental treatment plan may be generated during the course of a dental treatment for a patient. The dental treatment plan can comprise a three-dimensional model, such as a 3D mesh model or a 3D point cloud, that may be generated from a scan, such as an intraoral scan, of the patient's teeth. This dental treatment plan includes information that may be used to simulate, modify and/or choose between various orthodontic treatment plans. The feature alignment engine 178 is configured to add segmented 3D scan data (such as segmented data from a 3D CT scan, CBCT scan, or MRI scan) to be added to the 3D dental treatment plan. It is assumed that the 3D scan is segmented with software different than the dental treatment plan software, and the segmentation result is provided as 3D array of teeth and bone labels with scale information. The feature alignment engine automatically aligns segmented 3D scan data and populates the digital dental plan with realistic root and bone surfaces.

The feature alignment engine 178 can first produce a coarse alignment of segmented 3D scan data with the digital treatment plan. In one implementation, this coarse alignment can be based on a comparison of tooth crowns from the digital dental model with each corresponding segmented tooth volume from the segmented 3D scan. The feature alignment engine 178 can compute vectors from the center of jaw teeth to the center of opposite jaw teeth in the segmented 3D scan. Using these vectors, the system can find the most prominent “tip” point on each tooth of the segmented 3D scan. These “tip” points can be aligned with corresponding points in the digital dental treatment plan. The feature alignment engine 178 can then produce a fine alignment of the segmented 3D scan data with the digital treatment plan. This can be done with, for example, an iterative closest point (ICP) algorithm.

The bone preprocessing engine 180 is configured to provide preprocessing of 3D scan surfaces for reduction of digital noise and suppression of potential segmentation errors. In one implementation, the bone preprocessing engine 180 is configured to patch teeth sockets in the digital dental model. Once the teeth sockets are patched, semitransparent bone can be shown over planned teeth movement without visual interference of moving root contours and unmovable socket contours. This can be displayed to a user, for example, a user of a digital orthodontic treatment planning software. Socket zones can be detected as parts of bone surface which are close enough to some 3D scan teeth surfaces. Socket zones can be removed from bone surfaces by the bone preprocessing engine and the remaining holes can be filled with smooth patches. In one implementation, the bone preprocessing engine 180 can produce filtration of small, connected components and some generic smoothing of the surfaces.

The tooth numbering engine 182 can be configured to number and/or renumber individual teeth in the digital dental treatment plan. It should be noted that teeth numbering in the digital dental treatment plan and teeth numbering from the segmented 3D scan can be different. One typical reason is missed teeth. For example, if the first premolar is actually missing, automatic 3D scan segmentation can incorrectly guess that second premolar is missing instead of the first premolar. ICP surface matching can therefore be used to ignore teeth numbering and provide correct alignment for such cases. This process assumes that teeth numbering in the digital dental treatment plan are correct, and updates teeth numbering in the scanned 3D segmentation.

The merged dental model datastore 184 may be configured to store data related to the alignment between the segmented 3D scan and the digital dental model, the tooth/socket patching from the bone preprocessing engine, and/or the tooth numbering data from the modules described above.

FIG. 1D shows a diagram that includes an example of the tooth modeling engine(s) 164. The tooth modeling engine(s) 164 may include a generic tooth engine 186, a transformation engine 188, and a tooth modeling datastore 190. One or more of the modules of the tooth modeling engine(s) 164 may be coupled to each other or to modules not shown.

The generic tooth engine 186 may implement one or more automated agents configured to fit a generic tooth model into segmentation data from a segmented 3D scan. As described above, segmentation of 3D scans (such as CT scans, CBCT scans, MRI scans, etc.) can be performed on lower-resolution, resampled arrays, to fit the available fast and expensive memory on a GPU device. As a result, segmented details of the 3D scan can have noisy or low-resolution surfaces. To overcome these deficiencies in segmented 3D scans, the generic tooth engine 186 can be configured to use the segmentation data from the segmented 3D scan as auxiliary reference data, and fit into this data generic tooth models corresponding to the segmented teeth. Generic tooth models can be constructed, for example, in accordance with U.S. Pat. No. 7,844,429, which is incorporated herein by reference in its entirety. A generic tooth is a template of tooth of corresponding type (cuspid, incisor, premolar, etc.), and can be constructed in advance, using plurality of mesh-based models specific to that particular tooth, observed on different patients, and having special landmark points, (e.g., a set of 3D points that allow reconstructing the 3D mesh with desired resolution and characteristics, such as form, smoothness and so on). The generic tooth engine 186 can be configured to match an appropriate generic tooth model to the 3D scan segmentation data. In one implementation, the generic tooth engine can select a portion of a segmented 3D scan, such as an individually segmented tooth in the segmented 3D scan. The generic tooth engine can then select a corresponding generic tooth model to the selected tooth, and fit the generic tooth model into the segmentation data.

The transformation engine 188 may implement one or more automated agents configured to adjust the position and orientation of the generic tooth model to better match the position and orientation of the selected segmented tooth from the 3D scan data. The generic tooth model can be adjusted by adding or modifying several or all control points of the generic tooth model. In one implementation, adding control points can include finding apex positions in the segmentation data from the 3D scan for a particular tooth, overlaying contours of the generic tooth model onto the segmentation data, identifying discrepancies between the segmentation data and the generic tooth model, computing coordinates of points along the discrepancies, and adding one or more control points to the generic tooth model at these computed coordinates. The control points allow for the manipulation of the position/orientation of the generic tooth model. The adjusted generic tooth model can then be transformed into the segmented 3D scan (or into a digital dental treatment plan).

The tooth modeling datastore 190 may be configured to store data related to the data from the modules described above, including generic tooth model data, 3D control point data, and transformation data of the generic tooth model into the segmented 3D scan or into the digital dental treatment plan.

FIG. 1E shows a diagram that includes an example of the tooth labeling engine(s) 166. The tooth labeling engine(s) 166 may include an erosion engine 192 and a tooth labeling datastore 194. One or more of the modules of the tooth labeling engine(s) 166 may be coupled to each other or to modules not shown.

The erosion engine 192 may implement one or more automated agents configured to individually number/label segmented teeth in a segmented 3D scan. In one implementation, the erosion engine 192 receives as an input a binary volume of teeth (label map) received after automatic segmentation of a 3D scan (such as segmentation of a CT scan, a CBCT scan, or an MRI scan as described above). The erosion engine 192 may be configured to separate the label map with a watershed algorithm. In one implementation, seeds for the watershed algorithm are formed through iterations of erosion, applied to the label map. These seeds take into account the morphological structure of teeth during the seeds preparation, thus increasing the quality of volume separation. The seeds can be applied to the original binary label map, and the watershed algorithm can be applied again to segment the individual teeth into separate components for more accurate labeling.

The tooth labeling datastore 194 may be configured to store data related to the data from the modules described above, including the labeling/numbering data, erosion data, and seed data as described herein.

FIG. 1F shows a diagram that includes an example of the 2D alignment engine 169. The 2D alignment engine 169 may include a 3D model simulation engine 196, a 2D projection engine 197, a registration engine 198, and a registration datastore 199. The 2D alignment engine 169 determines an alignment between a patient's 2D dental image and a patient's 3D alignment model. The 3D alignment model is based on the patient's segmented 3D scan, determined as described herein. Therefore, the 3D alignment model may include some or all of the information from the patient's segmented 3D scan. For example, the 3D alignment model may include an upper jaw portion and/or a lower jaw portion. In some cases, the lower jaw portion may be positioned with respect to the upper jaw portion. The alignment between the 2D dental image and the 3D alignment model may be determined by generating a 2D projection of the patient's 3D alignment model and comparing the 2D projection to the patient's 2D dental image. If the 2D projection favorably compares to the patient's 2D dental image (i.e., the 2D projection matches the 2D dental image with a threshold), then information from the 3D alignment model (the segmented 3D model) may be used with (imported to) the 2D dental image.

A 2D projection of the patient's 3D alignment model may include a projection of one or more teeth. For example, the 2D projection may include incisor teeth numbers 7, 8, 9, and 10. The patient's 2D dental image may also include incisor teeth 7, 8, 9, and 10. The 2D alignment engine 169 can determine how well the 2D projection of the incisor teeth matches the 2D dental image of the same incisor teeth. That is, the 2D alignment engine 169 (using the 2D projection engine 197 and the registration engine 198 described below) may determine how well outlines of the incisor teeth in the 2D projection match outlines of the incisor teeth of the 2D dental image. A matching alignment may be based on matching outlines between the 2D projection and the 2D dental image, within a threshold. Although illustrated with incisor teeth here, any elements of the 2D projection and the 2D dental image may be compared with each other to determine how well they match.

After an alignment (also referred to as a matching alignment, or more simply a match) is determined, information from the 3D alignment model may be imported to the 2D image. Example information that may be imported may include tooth number information, although any feasible information may be imported and/or transferred. In some variations, the information from the 3D alignment model can be used with data from the 2D dental image to generate photo-realistic oral images which may be used for patient consultation, education, or diagnostics.

The 3D alignment model may be determined with the tooth modeling engine(s) 164, the tooth labeling engine(s) 166, or any other feasible engines, modules, or procedures. The patient's 3D model alignment may include an upper jaw portion that includes an upper dental arch and a lower jaw portion that includes a lower dental arch. The upper jaw portion may be separate from the lower jaw portion. Although separate, the position of the upper jaw portion and the lower jaw portion may be related through the patient's temporomandibular joint. In other words, the lower jaw portion may be positioned with respect to the upper jaw portion based on the location of the temporomandibular joint.

Locating the upper jaw portion in 3D space may be described with six variables that correspond to six degrees of freedom. Six example variables may include x, y, z (coordinates in cartesian coordinate system) and θ, Ω, and φ, (pitch, yaw, and roll rotations). Locating the lower jaw portion in 3D space may be done with respect to the upper jaw portion and a projected position of the temporomandibular joint. Since the temporomandibular joint restricts the positions of the lower jaw portion, locating the lower jaw portion in 3D space may be simplified to two degrees of freedom (y and z, for example) with respect to the upper jaw portion. In some variations, locating the lower jaw portion in 3D space may be simplified to one degree of freedom (an angle of rotation about the temporomandibular joint between the upper jaw portion and the lower jaw portion.

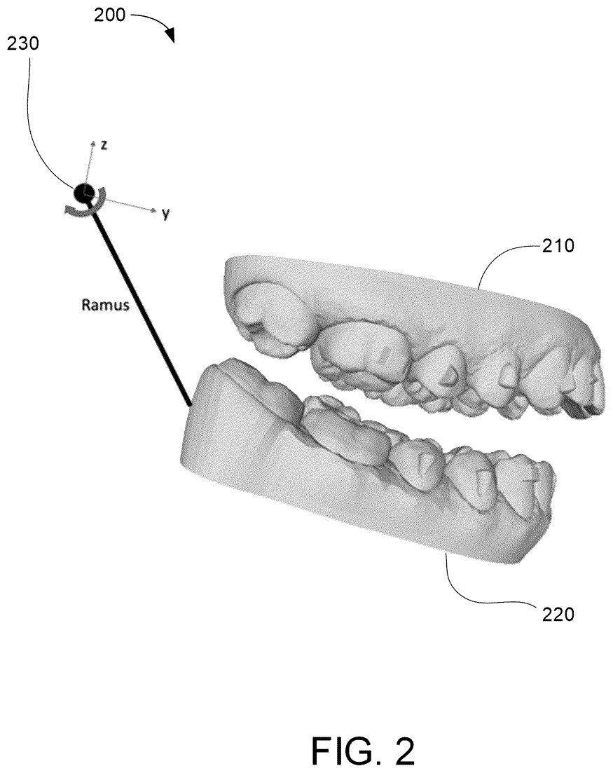

FIG. 2 shows an example 3D representation of a patient's dentition 200. The dentition 200 includes an upper jaw portion 210 and a lower jaw portion 220. As described above, the lower jaw portion 220 may be located with respect to the upper jaw portion 210 using a temporomandibular joint 230. In some variations, the upper jaw portion 210 may be located in 3D space using six variables (six degrees of freedom) and the lower jaw portion 220 can be located with small translational movements along two (y and/or z axis, for example) with respect to the upper jaw portion 210.

Notably, the converse may also be used to position the patient's dentition 200. That is, the lower jaw portion 220 may be located in 3D space using six variables and the upper jaw portion 210 is then located with respect to the lower jaw portion 220 using the temporomandibular joint 230.

Furthermore, positioning of the lower jaw portion 220 with respect to the upper jaw portion 210 may further include a jaw angle. The jaw angle describes the amount of jaw opening between the upper jaw portion 210 and the lower jaw portion 220. The jaw angle (based on the temporomandibular joint 230) may be a minimum angle (representing a closed mouth) to an arbitrary maximum angle (representing a fully open mouth).

Returning to FIG. 1F, the 3D model simulation engine 196 determines a position of the patient's 3D alignment model in a virtual 3D space. Thus, the 3D model simulation engine 196 locates the position of the patient's segmented 3D scan (which may include an upper jaw portion and a lower jaw portion) in 3D space. Determining the position of the patient's segmented 3D scan may include determining the position of an upper jaw portion using six variables (six degrees of freedom) and determining the position of the lower jaw portion using one or two variables (one or two degrees of freedom). The 3D model simulation engine 196 can also determine a jaw angle between the upper jaw portion and the lower jaw portion.

The 2D projection engine 197 can generate a 2D projection of the upper jaw portion and the lower jaw portion as positioned in 3D space by the 3D model simulation engine 196. The 2D projection is determined from the point of view of a virtual camera positioned in 3D space. The positioning of the virtual camera is determined, at least in part, by the location of the upper jaw portion and the lower jaw portion determined by the 3D model simulation engine 196. The 2D projection is projected onto a reference plane.

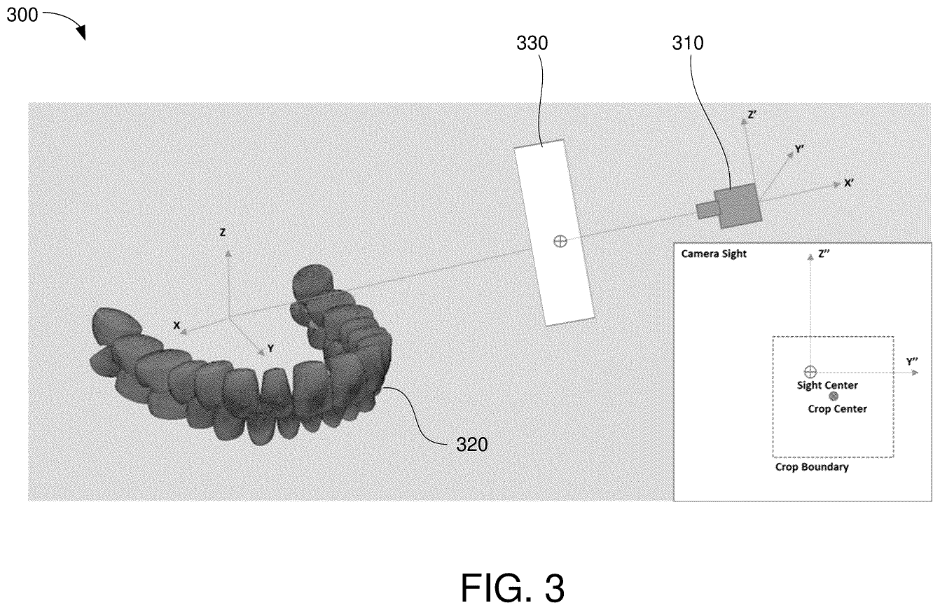

FIG. 3 shows an example virtual 3D space 300. The virtual 3D space 300 may be used to establish a relationship between a 3D alignment model and its related 2D projection. The 3D alignment model may include upper and lower jaw portions as well as a reference plane. Shown in the virtual 3D space 300 is a virtual camera 310, a lower jaw portion 320, and a reference plane 330. The virtual 3D space 300 shows only the lower jaw portion 320 for simplicity. In other variations, the virtual 3D space 300 may include both the upper jaw portion (not shown) and the lower jaw portion 320. The lower jaw portion 320 may be positioned in the virtual 3D space 300 based on the data from the 3D model simulation engine 196 and/or the position of the virtual camera 310. The reference plane 330 is an imaginary plane between the virtual camera 310 and the lower jaw portion 320. Typically, the reference plane 330 is perpendicular to the virtual camera 310. The virtual 3D space 300 is virtual since the patient's jaw and the virtual camera 310 may exist in a common, virtual 3D space. That is, the virtual 3D space 300 may be a construct used to determine or calculate one or more elements while not requiring any the existence of any physical space.

The 2D projection engine 197 can generate the 2D projection of the 3D alignment model onto the reference plane 330, in some cases by determining a visibility of any point on the 3D alignment model at the reference plane 330. In some embodiments, the 2D projection engine 197 can determine whether one or more points on the 3D alignment model are partially or totally occluded by other parts of the 3D alignment model from the point of view of the reference plane 330 and/or the virtual camera 310.

The registration engine 198 compares the 2D projection of the 3D alignment model to a 2D dental image of the patient. If the 2D projection matches the 2D dental image (within a comparison threshold), then the registration engine 198 can import or transfer corresponding information from the 3D alignment model (and/or the segmented 3D model providing a basis for the 3D alignment model) to the 2D dental image. As described above, any dental elements or features may be compared such as tooth outlines or the like to determine whether the 2D projection matches the 2D dental image. If a match is determined, then teeth number information, for example, may be copied or exported from the 3D alignment model to the 2D dental image.

On the other hand, if the 2D projection of the 3D alignment model does not match the 2D dental image, then the registration engine 198 can adjust the virtual camera position (through the 2D projection engine 197), the position of the 3D alignment model in 3D space and/or a relationship between the upper jaw portion and the lower jaw portion (through the 3D model simulation engine 196). After the position adjustment, the 2D projection engine 197 can generate a new (updated) 2D projection and the registration engine 198 can compare the new 2D projection to the 2D dental image. The registration engine 198 can iterate the process of positioning the 3D alignment model and/or the virtual camera until a match is detected. After the match is detected, data may be stored in the registration datastore 199. For example, the registration engine 198 can store 2D projection data as well as additional data (tooth number information, and the like) in the registration datastore 199.

In some examples, a portion of the projection plane 330 may be cropped prior to determining whether a match exists between the 2D projection and the 2D dental image. The cropping may help ensure that only relevant portions of each image is compared. The cropping may advantageously reduce any additional computations needed to determine a match.

The methods and apparatuses for performing them described herein may use a trained neural network (trained machine learning agent) to segment and/or number the teeth of the image and/or to compare one or more 3D images to one or more 2D images. For example, one or more original images of the patient's teeth may be segmented and/or renumbered using a trained neural network, and one or more images may be extracted from a 3D model of the patient's jaw(s) and rendered for comparison, in some cases by projecting an image for comparison with the segmentation mask. The methods and apparatuses may optimize the model(s) by minimizing the differences between the projection(s) and the segmentation mask. Rendering of one or more images from the 3D model(s) may be performed using camera parameters that may be initially set to a set of predetermined values as an initial starting set (e.g., initial guess); this initial set may be based on historical data, patient data, etc. The camera values may orient the 3D model in a 3D space, allowing virtual selection of one or more images of 3D model based on the camera parameters. The camera parameters may be refined by matching between the virtual (simulated) 2D image(s) and the actual image of the patient's teeth. As discussed above in reference to FIG. 3, the methods described herein may take a virtual image of the patient's teeth (e.g., jaws) and may crop the simulated image based on the camera parameters (e.g., may project a mesh onto an image plan indicating the center, e.g., camera optical axis, and may crop the image based on the camera parameters). Thus, the camera information may be used to prepare the 2D projections from the 3D model(s) including the upper and lower teeth; in addition to cropping the images, the camera settings may provide for rotation and perspective changes, including for generating a 2D image of the 3D model, which may generate a unique image of the upper and lower arch that may be compared to an actual image of all or portion of the upper and lower arch. For example, the camera parameters may be used to generate a virtual image (that may be segmented and/or have the teeth numbered) which may be compared directly to an image of the patient's teeth (which may also be segmented and/or numbered).

In general, the methods and apparatuses described herein may introduce a constraint the defines how the upper jaw and lower jaw move relative to each other. This constraint may be the TMJ relationship, e.g., the description of the patient's TMJ in terms of the upper and lower jaw, and in some cases in terms of the digital model of the upper and lower jaw, which defines how the two may move relative to each other. This approach is in contrast to earlier work which allowed unconstrained, or less realistically constrained, movement between the models of the upper and lower jaws (including virtual models).

The constraints on the TMJ may be modeled after the operation of the patient's anatomical TMJ, and may account for jaw shape (length), jawbone and/or muscle insertion site, etc.

In some examples, a standard model for how the patient's TMJ operates may be used. Projections of the mandibular bone (e.g., typically between 2-6 inches, e.g., approximately 4 inches back from the molars) may be hinged relative to the upper jaw, around the TMJ. The lower jaw typically hinges at the TMJ so that the lower jaw may move relative to the upper jaw, rotating around the TMJ. Other models of jaw movement and the TMJ may account for other, often limited, freedom of movement about the TMJ. For example, the upper jaw may be considered ground relative to movement of the lower jaw, which may be free to move in a constrained manner relative to the upper jaw; the lower jaw may have 6 or 7 degrees of freedom, although the overall degrees of freedom may be limited or constrained (e.g., by limiting the amount of front/back, side-side, and pivoting open/close relative to the upper jaw). The methods of modeling the TMJ described herein may allow determination of where the jaws are positioned relative to each other. Although the lower jaw may move independently of the upper jaw, or relative to the upper jaw, the constraints of the TMJ relationship may prevent or limit the relative locations of the upper and lower jaws. Thus, knowing the position of the upper jaw may allow derivation of the position of the lower jaw within the constrained movement range; once the upper jaw is used as a reference, there are only 1-2 degrees of freedom (DOF) that could indicate where the lower jaw is; specifically, how “open” is the mouth.

Thus, in practice, these methods and apparatuses may set up the 3D models of the upper and lower jaw and render one or more 2D images of the combined upper and lower jaw with the jaws open/closed to various degrees that may be derived from the TMJ relationship, with respect to a camera (e.g., a set of camera parameters). This may allow the apparatus to render the upper and lower jaw with respect to the camera to determine how to adjust the model and/or camera position. Because the position of the lower jaw is limited with respect to the upper jaw, the lower jaw position may be estimated from the upper jaw position. Similarly, the TMJ relationship may be derived by applying constrains to the lower jaw position relative to the upper jaw position and solving for the parameters defining these constraints using actual images of the patient's upper and lower jaws in various positions (e.g., open, closed, or any intermediate position, including positions associated with talking, cating, etc.).

For example, the TMJ parameters may be related to the constraints on the upper and lower jaw (and TMJ) digital model(s). In some examples, at least one parameter of the TMJ parameters may include the location of the hinge between the upper and lower jaws, which may be a fixed distance from lower jaw or may vary within a limited range. This parameter may be adjusted or optimized. In some examples the location of the hinge/joint region may be fixed relative to the upper jaw in the 3D modeling of the combined model including the upper and lower jaws.

The digital 3D model of the patient's upper and lower jaws may be taken (e.g., by intraoral scanning, by scanning a dental impression, etc.) either separately or together. The location of the TMJ (e.g., hinge joint) may then be determined and added to the digital model. The location of the TMJ may be initially unknown, but may be assumed using an initial (starting) value that may be an initial preset value(s), may be selected from a database (e.g., library) of possible values based on one or more properties of the patient (size, gender, arch dimension, etc.), or it may be solved for using an approximation based on one or more properties of the patient. An initial set of values for the TMJ parameters may then be refined to more accurately model the upper jaw, lower jaw and TMJ.

For example, the method or apparatus may determine or refine the location of the TMJ hinge base on how the patient moves her or his jaw (open/close the mouth) based on images or video data. In some examples the method and/or apparatus may determine the position of the upper jaw and lower jaw and TMJ in (virtual) 3D space; a projection of the 3D model may be made using the camera or image plan data (e.g., using camera parameter), to give a rendered image. The projection from the 3D model using the camera parameters may be compared to one or more actual (e.g., “original”) images, e.g., photographs, or to an image mask, such as a segmentation image mask, as described herein. The difference between the projection (the synthesized image) and the mask may be minimized by adjusting one or more parameters, which may be constrained by the constraints on the TMJ parameters. For example, difference may be minimized by adjusting the angle between the upper and lower jaws and/or jaw length, etc. This process may be iterative, so that multiple projections may be taken using the camera parameters following adjustments of one or more TMJ parameters, in order to determine the TMJ parameters. This process may be performed in parallel or sequentially (or in some cases, concurrently) for multiple open/closed positions. The difference between the projection and the actual images may be minimized for the set of parameters until the difference(s) falls below a threshold level (or fails to converge after a predefined limit). Once the rendered image matches the input (actual) photos within the difference threshold, the TMJ parameters may be considered to be solved. Each set of camera parameters and jaw (TMJ) parameters may be optimized separately or concurrently. The coordinate system may not matter, and may be used in terms of polar, spherical, cylindrical and/or cartesian coordinates. In some cases the jaw position may be set or specified based, at a minimum, on an angle of the TMJ and on the location of the TMJ. The set of TMJ parameters may be defined by jaw length and/or angular path.

In general, the methods and apparatuses described herein may determine the TMJ parameters by either solving for them and/or by measuring them from a segmented CBCT. For example, TMJ parameters may be measured directly from images (including but not limited to CBCT images, 2D X-ray images, other tissue-penetrative scans, etc.). The measurements may be used instead of or in addition to the parameters determined as described herein.

In any of the method and apparatuses described herein, the TMJ parameters may be determined or configured using at one image taken of the patient's upper and lower jaw with a closed bite and an open bite; in some cases two or more images for each jaw position may be used, and two or more jaw positions (open, closed, first intermediate open position, second intermediate open position, etc.) may be used. Multiple camera positions may be used. Once the relationship between the upper and lower jaw have been determined, which may be referred to as the TMJ relationship, the parameterization between the lower and upper jaw may be determined, defining the constrained degrees of freedom (e.g., 1 or 2 for the TMJ length and angle). Thus, it may be helpful to know or determine the location of the lower jaw with respect to camera and how open or close the patient's bite is, including identifying a deep bitc.

For example, a method as described herein may include receiving or taking one or more actual images of the patient's teeth (including both upper and lower jaws, or at least portions of both), and segmenting these actual images. Segmentation may include forming a segmentation mask. Any appropriate technique for segmenting and/or forming a segmentation mask may be used. One or more projected images may be taken from a 3D model of the patient's dental arch including the upper and lower jaws having a TMJ parameter or set of parameters; the image may be rendered using camera parameters that approximate the camera parameters (e.g., position, orientation, etc.) of the one or more actual images. The projected (e.g. rendered) images may then be compared to the original images, cither directly or, preferably, by comparing to the segmentation mask. If the comparison is sufficiently close (e.g., within a threshold range), the TMJ parameter(s) may be considered accurate; if not, the TMJ parameter(s) may be adjusted, new projections taken, and the process repeated until the error between the actual and projected images is within the target range (threshold range). Once within the threshold range, the final registration (the final TMJ parameter(s)) may be output, and the overall model of the patient's upper and lower jaws and dentition may be used.

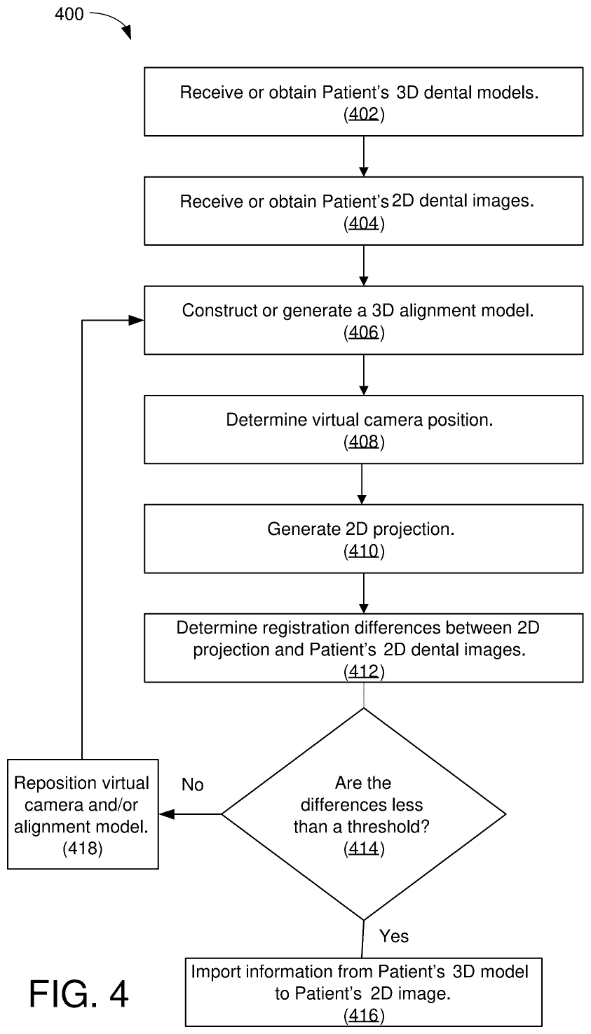

FIG. 4 is a flowchart showing an example method 400 for determining a match between a 2D projection and a 2D dental image and importing dental information from a 3D alignment model to the corresponding 2D dental image. Some examples may perform the operations described herein with additional operations, fewer operations, operations in a different order, operations in parallel, and some operations differently. The method 400 is described below with respect to the computing environment 100 of FIG. 1, however, the method 400 may be performed by any other suitable system or device.

In this example, the method 400 may initially receive or obtain a patient's 3D dental model 402. The 3D dental model may include some or all data from a patient's 3D scan (received from an intraoral scanning system, such as the scanning system 154 of FIG. 1). In some cases, the data may include segmentation information (e.g., received from the scan segmentation engine(s) 160). The 3D dental model may be associated with a patient's previously determined dental treatment plan. In some examples, the 3D dental model may include an upper jaw portion, a lower jaw portion, and a location of a temporomandibular joint (e.g., TMJ parameter(s)). The segmentation information may include any feasible dental information such as tooth numbers, tooth outlines, gingiva location, and the like.

Next in block 404, a patient's 2D dental image may be received or obtained. The 2D dental images may be taken or captured at a different time with respect to the 3D dental model of block 402. For example, the 2D dental images may have been captured to track the progress of a dental treatment program. The 2D dental images may be captured by the scanning system 154, a camera or smartphone, or any other feasible device.

Next in block 406, a 3D alignment model is constructed or generated. The 3D alignment model may be generated by the 3D model simulation engine 196. The 3D alignment model may include one or more features of the 3D dental model received in block 402. Construction or generation of the 3D alignment may include an upper jaw portion and a lower jaw portion positioned with respect to each other based on the location of a temporomandibular joint. In some examples, construction or generation of the 3D alignment model may include determining a relationship between portions of the 3D dental model. For example, the 3D model simulation engine 196 may determine a relationship between the upper jaw portion and the lower jaw portion of the patient's 3D dental model. In some cases, the 3D model simulation engine 196 may determine a relationship between the upper jaw portion and the lower jaw portion based on degrees of freedom between the 3D dental model and 3D space.

Next in block 408, a virtual camera position is determined. The virtual camera position establishes a relationship between the 3D alignment model and a 2D image. That is, a 2D image or projection can be generated from the 3D alignment model based on a point-of-view associated with the position of the virtual camera (e.g., camera 310 of FIG. 3). By determining the position of the virtual camera, the position of a projection plane (e.g., projection plane 330) may be determined. Generally, the projection plane is perpendicular to the virtual camera and disposed between the camera and the 3D alignment model.

Next in block 410, a 2D projection is generated. The 2D projection (sometimes called a 2D alignment projection) may be generated or determined by the 2D projection engine 197. In some implementations, the 2D projection engine 197 can draw rays from the 3D alignment model to the projection plane. The 2D projection engine 197 can determine which parts of the 3D alignment model are visible or occluded by elements including gingiva or any other feasible items.

Next in block 412, registration differences between a 2D projection and a 2D dental image are determined. In some examples, the registration differences may be determined by the registration engine 198. Registration differences may refer to one or more detectable or measurable differences between the 2D projection and the 2D dental image. For example, the registration engine 198 can determine differences between tooth outlines of corresponding teeth included in the 2D projection and the 2D dental image. Although tooth outlines are used as an example here, the registration engine 198 can determine difference between other corresponding items, such as bones, gingiva, or the like.

Next, in block 414, the determined differences are compared to a threshold. For example, a measurement may be made to determine a difference between outlines of corresponding teeth of a 2D projection and a 2D dental image. If the determined differences (e.g., if the measurement between outlines) is less than a threshold, then in block 416 information from the patient's 3D alignment model (which can include segmentation information) can be imported or transferred to the patient's 2D dental image. For example, tooth number information can be imported from the patient's 3D alignment model into the patient's 2D dental image. The imported dental information may be stored in the registration datastore 199. In some implementations, the 2D alignment engine 169 can generate a 3D model or image based on the 3D alignment model and/or the 2D dental image. Alternatively or additionally, the 3D model, including the TMJ parameter that has been refined by this method, may be output; the final 3D model may include the upper jaw, lower jaw and TMJ relationship (e.g. position).

Returning to block 414, if the determined differences are greater than a threshold, then in block 418 a new position of the virtual camera is determined and/or a new position of the 3D alignment model is determined. Since the determined differences are greater than a threshold, the correspondence between the 3D alignment model and the 2D dental image may be significant. In order to try to reduce the differences, the 3D model simulation engine 196 can determine a new 3D alignment model and/or a new virtual camera position may be determined. After determining a new 3D alignment model and/or a new virtual camera position, the method returns to block 406 to iteratively determine if the a 2D projection of the new 3D alignment model is similar to (matches) the 2D dental image. The alignment model may, in some examples, refer to the TMJ parameter, such as TMJ position (jaw length, etc.). In any of these methods and apparatuses, two TMJ joints may be used or a single TMJ joint may be sufficient.

Any of these methods may include determining a 3D model that includes the patient's upper jaw, the patient's lower jaw, and a TMJ parameter. The TMJ parameter may be optimized as described herein and may inform how the lower jaw may move (e.g., hinged at an angle relative to the upper jaw, as well as in 2 translational directions, e.g., y, z). The position or location of the TMJ(s) (the TMJ parameter) relative to the upper and/or lower jaw may be determined iteratively. Thus, the 3D models described herein including the upper jaw, lower jaw, and parameters (TMJ parameters) may be used to predict how the lower jaw may move for a particular patient based on the 2D registration as described herein.

For example, any of these methods an apparatuses may include starting with one or more intraoral scans of the patient's upper and lower jaws (upper and lower arches). The relationship between the upper and lower jaws may be defined by one or more parameters, including a modeled temporomandibular joint (TMJ(s)). In general, these methods and apparatuses may determine, using 2D to 3D registration of a plurality of images of the patient's upper and lower jaws, an estimated location for the modeled TMJ. Once identified, the jaw parameters, including in particular the modeled TMJ, may be used move the 3D modeled lower jaw relative to the 3D modeled upper jaw with the limited constrained degrees of freedom, e.g., rotation of the lower jaw model about the modeled TMJ, and limited movement in x, y, and/or z (e.g., in some cases, y and z).

Thus, the relationship between the modeled upper jaw, modeled lower jaw and modeled TMJ may be identified by setting an initial positions (e.g., guesses) of the modeled TMJ and lower jaw position relative to the ‘ground’ upper jaw, generating one or more 2D projections including the upper and lower jaw based on this best-guess position and comparing them to actual 2D images to determine how different the positions are. The parameters (e.g., TMJ position and/or constrains, x, y, z constraints, etc.) may be adjusted and the lower jaw position iteratively changed to better match the actual 2D image(s), until the differences between the 2D projections generating using the parameters and the actual 2D images (which may be compared using the segmentation in some examples) is below a threshold difference (e.g., converges within a threshold) or the number of iterations exceeds a maximum threshold. The threshold may be set by the user or system and may be adjustable. Thus, these methods may determine the parameters that act as constraints that indicate how the upper and lower jaw move relative to each other.

These methods and apparatuses may a simple models for how the hinging of the TMJ operates between the upper and lower jaws (e.g., the TMJ may be expected to be approximately 4 inches behind the lower jaw, etc.). Any appropriate model for the operation of this hinging by the TMJ(s) may be used. These methods may generally use the upper jaw as ‘ground’ about which the lower jaw may rotate and move in limited x, y movement. This limits the degrees of freedom of the lower jaw to 2-3 degrees of freedom that could indicate where the lower jaw is located as the mouth is opened/closed. Thus, in practice the methods and apparatuses described herein may receive the 3D models of the upper and lower jaws, which in some cases may be segmented, and may iteratively solve for the parameters (e.g., TMJ parameters, including TMJ position(s)/locations(s) relative to the upper and lower jaw) by comparing to 2D images from multiple different camera positions. The TMJ parameter(s), which may indicate the location of the TMJ(s) (e.g., a single TMJ in some examples or left and right TMJs in some examples) may be optimized to determine the location of the “hinge” formed by the TMJ(s), which may be estimated as an optimization parameter. Initially, the position of the TMJ(s) may be estimated to assume an initial position for his hinge as one of the parameters for the model. Given the hinge (TMJ) position, the method may include moving the virtual model of the lower jaw relative to the upper jaw. The method may include setting a virtual camera in the 3D space relative to the upper and lower jaws. The method may also include using the 3D model to project from the camera onto the image plane, to generate a rendered 2D image from the 3D model. This projected image may be compared to an original photo or image mask taken at approximately the same camera position and/or orientation. The difference between the projected image taken of using the initial parameters (e.g., hinge/TMJ position) and the 2D image taken at approximately the same camera angle(s) may be used to adjust the parameters, including the TMJ parameter(s). this process may be repeated after adjusting parameters, moving the lower jaw relative to the upper jaw based on the updated/new parameters. With each set of parameter adjustment(s) the match between the rendered/projected 2D images and the actual 2D images may improve until, once the rendered 2D image matches the actual 2D image(s) closely enough, e.g., within a target range, the parameters may be finalized. In any of these methods multiple images may be used. In some cases each set of camera parameters and jaw parameters (e.g., TJM parameters) may be expressed as an angle and/or x, y, z and open/closed position of the jaws, etc. The quality of the fit of the parameters (e.g., TMJ location and/or range of angle, range of movements, etc.) may be determined as the quality (e.g., ‘goodness’) of the fit. Ideally, these methods and apparatuses may determine a set of parameters that best fit (e.g., have a maximum likelihood of matching 2D projections from the 3D model to 2D images taken with one or more cameras. In some cases, once the methods or apparatus determines a relationship between the upper and lower jaws (e.g., looking at a first image or set of images), the procedure may be repeated, e.g., reformulating for new parameterization.

As mentioned, the comparison between the 2D image and the 3D image may be based on segmentation or other landmarks between the 2D projected image and the actual 2D image. Once the relationship of upper and lower jaws is determined by the parameters, the 3D model, including the parameters, may be used to determine the positions of the jaws, and therefore the teeth and gingiva, in any virtual position, which may be extremely useful for identifying and/or treating dental conditions including, but not limited to, open bite and deep bite conditions.

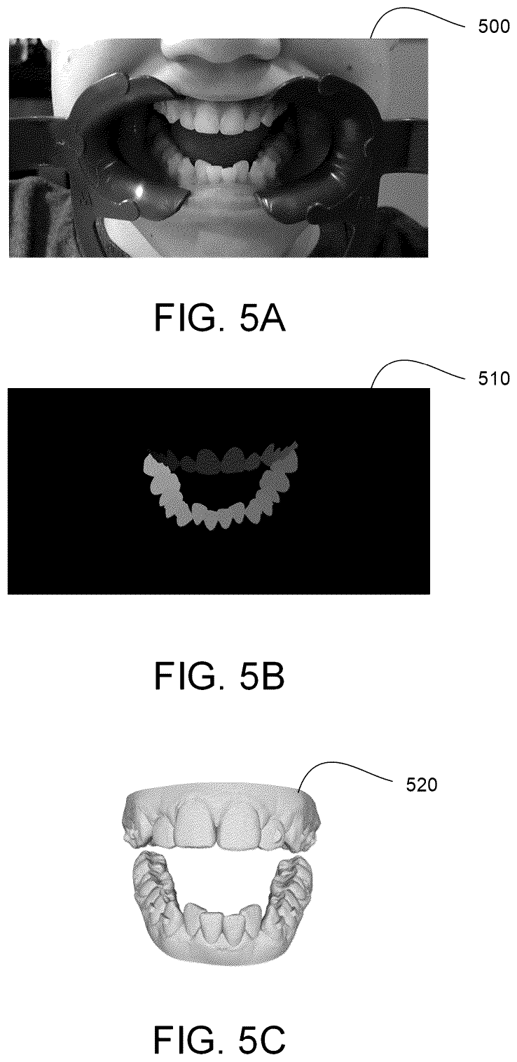

FIGS. 5A-5C show images associated with an open bite, as described herein. FIG. 5A shows a 2D dental image 500 of a patient's dentition. FIG. 5B shows a 2D projection of a jaw pair 510 based on the 2D dental image 500. The jaw pair 510 may include an upper and lower jaw portion and, in some cases, may be based on a projection of a 3D alignment model positioned in a 3D virtual space as described herein. FIG. 5C shows a 3D model 520 based on the 2D projection of the jaw pair 510. For example, the 2D projection of the jaw pair 510 may be favorably compared to (less than a threshold) the 2D dental image 500. The 3D model 520 may be generated based on the 2D projection of the jaw pair 510, the 2D dental image 500, or a segmented 3D dental model (not shown).

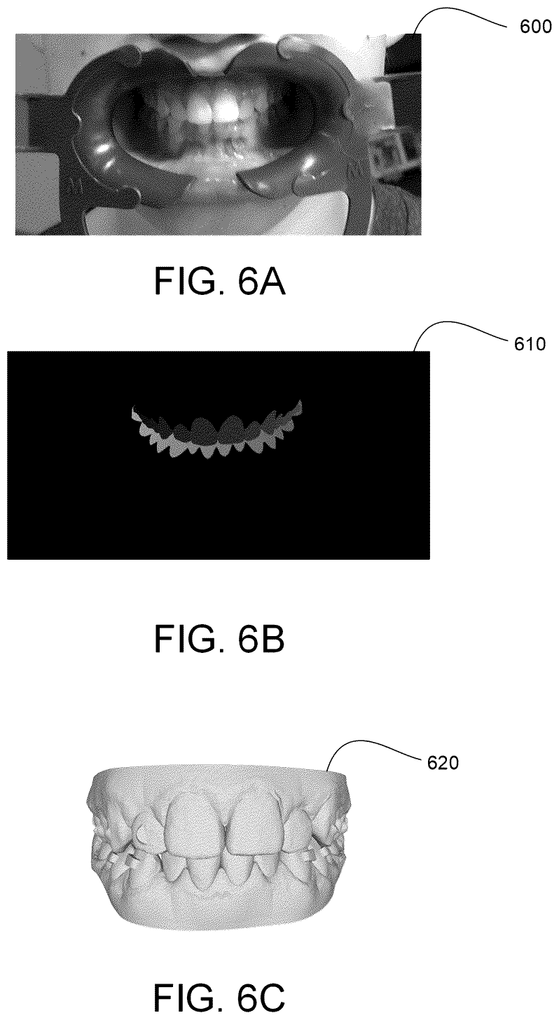

FIGS. 6A-6C show images associated with a closed bite, as described herein. FIG. 6A shows a 2D dental image 600 of a patient's dentition. FIG. 5B shows a 2D projection of a jaw pair 610 based on the 2D dental image 600. The jaw pair 610 may include an upper and lower jaw portion and, in some cases, may be based on a projection of an alignment model positioned in a 3D virtual space as described herein. FIG. 6C shows a 3D model 620 based on the 2D projection of the jaw pair 610. For example, the 2D projection of the jaw pair 610 may be favorably compared to (less than a threshold) the 2D dental image 600. The 3D model 620 may be generated based on the 2D projection of the jaw pair 610, the 2D dental image 600, or a segmented 3D dental model (not shown). In contrast to FIGS. 5A-5C, the FIGS. 6A-6C show a closed bite and how the closed bite can affect one or more images.

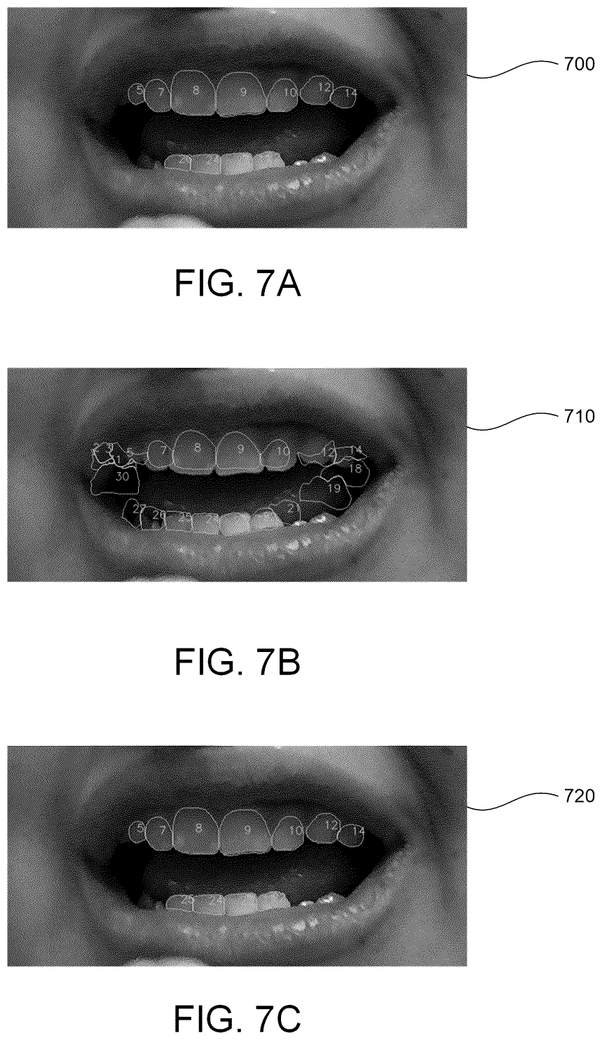

FIGS. 7A-7C show images associated with determining tooth numbers based on 2D dental images. FIG. 7A shows a first 2D dental image 700 where tooth numbers have been determined through machine language operations or procedures. In some instances, the tooth numbers may include errors. For example, tooth 26 is shown error. FIG. 7B shows a second 2D dental image 710 which includes one or more tooth numbers that may have been imported from a 3D alignment model and operations such as those described with respect to the example method 400. FIG. 7C shows a 2D dental image 720 with a corrected tooth numbers.

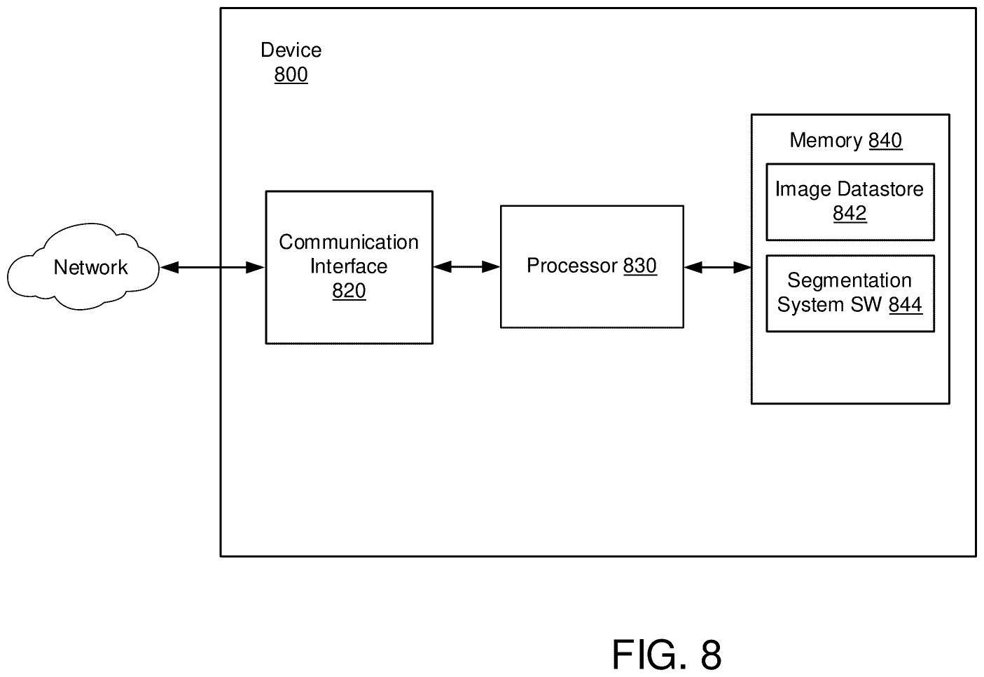

FIG. 8 is a block diagram of a device 800 that may be one example of a device configured to perform one or more operations described herein. The device 800 may include a communication interface 820, a processor 830, and a memory 840.

The communication interface 820, which may be coupled to a network and to the processor 830, may transmit data to and receive data from other wired or wireless devices, including remote (e.g., cloud-based) storage devices, cameras, processors, compute nodes, processing nodes, computers, mobile devices (e.g., cellular phones, tablet computers and the like) and/or displays. For example, the communication interface 820 may include wired (e.g., serial, ethernet, or the like) and/or wireless (Bluetooth, Wi-Fi, cellular, or the like) transceivers that may communicate with any other feasible device through any feasible network. In some examples, the communication interface 820 may receive previous dental data (including treatment plans) and/or current dental data.

The processor 830, which is also coupled to the memory 840, may be any one or more suitable processors capable of executing scripts or instructions of one or more software programs stored in the device 800 (such as within memory 840).

The memory 840 may include an image datastore 842 that may be used to locally store patient image data. For example, the image datastore 842 may include the segmented model datastore 167 of FIG. 1A, the registration datastore of FIG. 1F, or any other feasible information.

The memory 840 may also include a non-transitory computer-readable storage medium (e.g., one or more nonvolatile memory elements, such as EPROM, EEPROM, Flash memory, a hard drive, etc.) that may store segmentation system software 844. The segmentation system software 844 may include program instructions that, when executed by the processor 830, may cause the device 800 to perform the corresponding function(s). Thus, the non-transitory computer-readable storage medium of memory 840 may include instructions for performing all or a portion of the operations described herein.

Execution of the segmentation system software 844 may cause the processor 830 to perform operations of the scan segmentation engine(s) 160, the tooth labeling engine(s) 166, and/or the 2D alignment engine 169. For example, the processor 830 may execute the segmentation system software 844 to process, obtain, and/or receive 2D and 3D dental images as well as segment dental images to determine separate parts of a patient's dentition such as individual teeth, gingiva, and the like. In some implementations, executing the segmentation system software 844 may determine and identify upper jaw portions and lower jaw portions of a patient.

The processor 830 may execute the segmentation system software 844 to position 3D dental models in 3D space. Positioning 3D dental models may include determining a relative position of upper and lower jaw portions. Execution of the segmentation system software 844 may determine a 2D projection associated with any 3D dental models.

The processor 830 may execute the segmentation system software 844 to determine and label teeth with corresponding tooth numbers. The processor 830 may execute the segmentation system software 844 to determine a 2D projection of 3D dental models and determine if the 2D projection matches a 2D dental image. In some variations, execution of the segmentation system software 844 can import or transfer data from the segmented 3D dental model to the 2D projection.

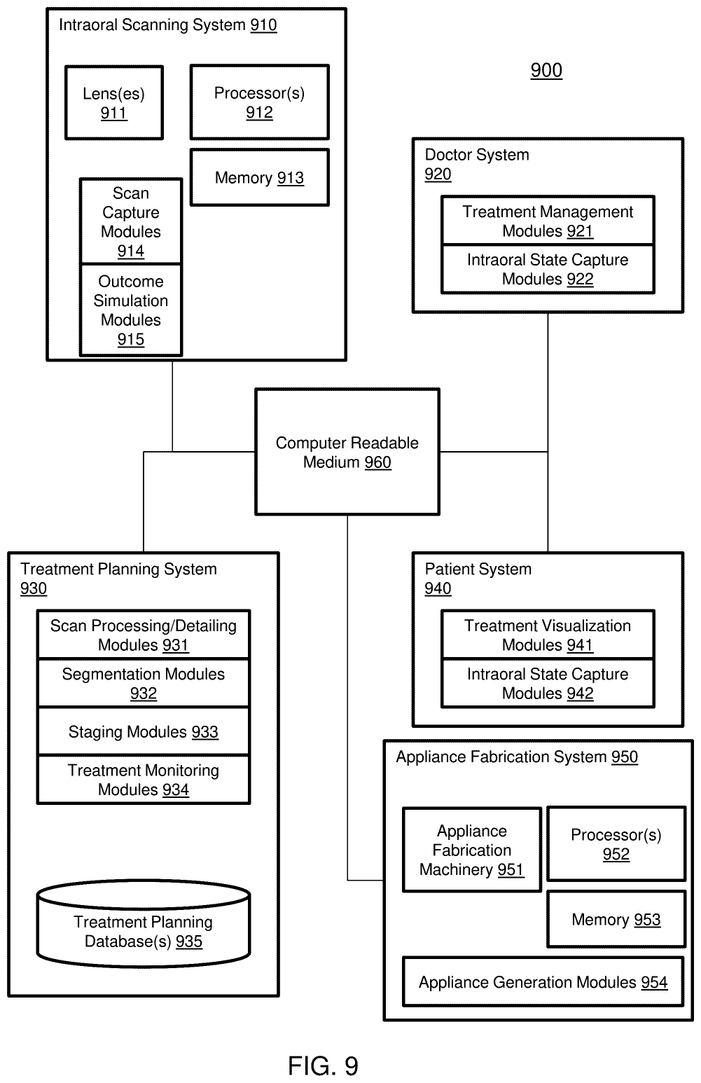

In general, these methods and apparatuses may be used at one or more parts of a dental computing environment, including as part of an intraoral scanning system, doctor system, treatment planning system, patient system, and/or fabrication system. In particular, these methods and apparatuses may be used as part of a treatment planning system, for example, to generate an accurate digital model of the patient's dentition, from which treatment plans, and/or designs for one or more dental appliances to perform the treatment plan may be generated. For example, FIG. 9 is a diagram illustrating one variation of a computing environment 900 that may generate one or more orthodontic treatment plans specific to a patient, and fabricate dental appliances that may accomplish the treatment plan to treat a patient, under the direction of a dental professional. The example computing environment 900 shown in FIG. 9 includes an intraoral scanning system 910, a doctor system 920, a treatment planning system 930, a patient system 940, an appliance fabrication system 950, and computer-readable medium 960. In some variations a computing environment (dental computing system) 900 may include just one or a subset of these systems (which may also be referred to as sub-systems of the overall system 900). Further, one or more of these systems may be combined or integrated with one or more of the other systems (sub-systems), such as, e.g., the patient system and the doctor system may be part of a remote server accessible by doctor and/or patient interfaces. The computer readable medium 960 may divided between all or some of the systems (subsystems); for example, the treatment planning system and appliance fabrication system may be part of the same sub-system and may be on a computer readable medium 960. Further, each of these systems may be further divided into sub-systems or components that may be physically distributed (e.g., between local and remote processors, etc.) or may be integrated.

An intraoral scanning system may include an intraoral scanner as well as one or more processors for processing images. For example, an intraoral scanning system 910 can include lens(es) 911, processor(s) 912, a memory 913, scan capture modules 914, and outcome simulation modules 915. In general, the intraoral scanning system 910 can capture one or more images of a patient's dentition. Use of the intraoral scanning system 910 may be in a clinical setting (doctor's office or the like) or in a patient-selected setting (the patient's home, for example). In some cases, operations of the intraoral scanning system 910 may be performed by an intraoral scanner, dental camera, cell phone or any other feasible device.

The lens(es) 911 include one or more lenses and optical sensors to capture reflected light, particularly from a patient's dentition. The scan capture modules 914 can include instructions (such as non-transitory computer-readable instructions) that may be stored in the memory 913 and executed by the processor(s) 912 to can control the capture of any number of images of the patient's dentition.

As mentioned, in some examples the methods and apparatuses described herein for generating a 3D model including both the upper jaw, lower jaw and TMJ may be part of, or accessible by, the intraoral scanning system 910, computer readable medium 960 and/or treatment planning system 930.

For example, the outcome simulation modules 915, which may be part of the intraoral scanning system 910, can include instructions that simulate the tooth positions based on a treatment plan. In some cases, the outcome simulation modules 915 can include instructions that simulate tooth positions based on a position of a temporomandibular joint as described above with respect to FIGS. 2 and 3. The temporomandibular joint may enable a lower jaw portion to be simulated with respect to an upper jaw portion. The lower jaw portion may be located with respect to the upper jaw portion with six degrees of freedom.

Alternatively or additionally, in some examples, the outcome simulation modules 915 can import tooth number information from 3D models onto 2D images to assist in determining an outcome simulation as described above with respect to FIG. 4.

Any of the component systems or sub-systems of the dental computing environment 900 may access or use the 3D model of the patient's dentition generated by the methods and apparatuses described herein. For example, the doctor system 920 may include treatment management modules 921 and intraoral state capture modules 922 that may access or use the 3D model including the upper jaw, lower jaw and TMJ. The doctor system 920 may provide a “doctor facing” interface to the computing environment 900. The treatment management modules 921 can perform any operations that enable a doctor or other clinician to manage the treatment of any patient. In some examples, the treatment management modules 921 may provide a visualization and/or simulation of the patient's dentition with respect to a treatment plan. For example, the doctor system may include a user interface for the doctor that allows the doctor to manipulate the 3D model including the upper jaw, lower jaw and TMJ, including moving the upper and lower jaws relative to each other based on the TMJ, after accurately configuring the patient's TMJ.

The intraoral state capture modules 922 can provide images of the patient's dentition to a clinician through the doctor system 920. The images may be captured through the intraoral scanning system 910 and may also include images of a simulation of tooth movement based on a treatment plan.

In some examples, the treatment management modules 921 can enable the doctor to modify or revise a treatment plan, particularly when images provided by the intraoral state capture modules 922 indicate that the movement of the patient's teeth may not be according to the treatment plan. The doctor system 920 may include one or more processors configured to execute any feasible non-transitory computer-readable instructions to perform any feasible operations described herein.

Alternatively or additionally, the treatment planning system 930 may include any of the methods and apparatuses described herein, and/or may access the results (e.g., a 3D model including both the upper jaw, lower jaw and TMJ. The treatment planning system 930 may include scan processing/detailing modules 931, segmentation modules 932, staging modules 933, treatment monitoring modules 934, and treatment planning database(s) 935. In general, the treatment planning system 930 can determine a treatment plan for any feasible patient. The scan processing/detailing modules 931 can receive or obtain dental scans (such as scans from the intraoral scanning system 910) and can process the scans to “clean” them by removing scan errors and, in some cases, enhancing details of the scanned image.

The treatment planning system 930 may include a segmentation system (as shown in FIG. 1A) or it may access a segmentation system and/or may include any of the engines (e.g., scan segmentation engine(s), 3D fusion engine(s), tooth modeling engine(s), tooth labeling engine(s), and/or 2D alignment engine) described in FIGS. 1B-1F.

A treatment planning system may include a segmentation modules 932 that can segment a dental model into separate parts including separate teeth, gums, jaw bones, and the like. In some cases, the dental models may be based on scan data from the scan processing/detailing modules 931.

The staging modules 933 may determine different stages of a treatment plan. Each stage may correspond to a different dental aligner. The staging modules 933 may also determine the final position of the patient's teeth, in accordance with a treatment plan. Thus, the staging modules 933 can determine some or all of a patient's orthodontic treatment plan. In some examples, the staging modules 933 can simulate movement of a patient's teeth in accordance with the different stages of the patient's treatment plan.

The treatment monitoring modules 934 can monitor the progress of an orthodontic treatment plan. In some examples, the treatment monitoring modules 934 can provide an analysis of progress of treatment plans to a clinician. The orthodontic treatment plans may be stored in the treatment planning database(s) 935. Although not shown here, the treatment planning system 930 can include one or more processors configured to execute any feasible non-transitory computer-readable instructions to perform any feasible operations described herein.

The patient system 940 can include treatment visualization modules 941 and intraoral state capture modules 942. In general, the patient system 940 can provide a “patient facing” interface to the computing environment 900. The treatment visualization modules 941 can enable the patient to visualize how an orthodontic treatment plan has progressed and also visualize a predicted outcome (e.g., a final position of teeth). In some examples, the treatment visualization modules 941 may use a position of a temporomandibular joint to determine upper and lower jaw positions, as described with respect to FIGS. 2 and 3.

In some examples, the patient system 940 can capture dentition scans for the treatment visualization modules 941 through the intraoral state capture modules 942. The intraoral state capture modules can enable a patient to capture his or her own dentition through the intraoral scanning system 910. Although not shown here, the patient system 940 can include one or more processors configured to execute any feasible non-transitory computer-readable instructions to perform any feasible operations described herein.

The appliance fabrication system 950 can include appliance fabrication machinery 951, processor(s) 952, memory 953, and appliance generation modules 954. In general, the appliance fabrication system 950 can directly or indirectly fabricate aligners to implement an orthodontic treatment plan. In some examples, the orthodontic treatment plan may be stored in the treatment planning database(s) 935.

The appliance fabrication machinery 951 may include any feasible implement or apparatus that can fabricate any suitable dental aligner. The appliance generation modules 954 may include any non-transitory computer-readable instructions that, when executed by the processor(s) 952, can direct the appliance fabrication machinery 951 to produce one or more dental aligners. The memory 953 may store data or instructions for use by the processor(s) 952. In some examples, the memory 953 may temporarily store a treatment plan, dental models, or intraoral scans.

The computer-readable medium 960 may include some or all of the elements described herein with respect to the computing environment 900. The computer-readable medium 960 may include non-transitory computer-readable instructions that, when executed by a processor, can provide the functionality of any device, machine, or module described herein.

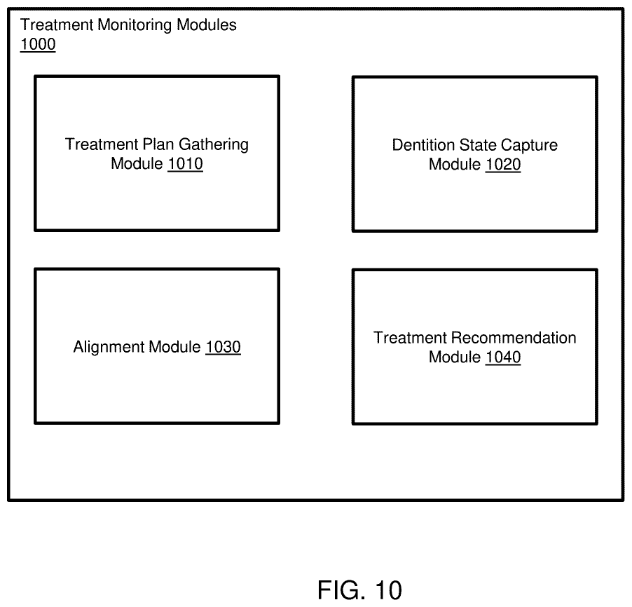

FIG. 10 is a block diagram of one example of treatment monitoring modules 1000.

The treatment monitoring modules 1000 may be an example of the treatment monitoring modules 934 of FIG. 9. The treatment monitoring modules 1000 may include a treatment plan gathering module 1010, a dentition state capture module 1020, an alignment module 1030, and a treatment recommendation module 1040. In general, the treatment monitoring modules 1000 can enable a clinician to monitor a patient's progress with respect to a treatment plan.

The treatment plan gathering module 1010 can retrieve a patient's treatment plan. In some cases, the patient's treatment plan may be stored in, and retrieved from, the treatment planning database(s) 935. The dentition state capture module 1020 can capture or obtain any feasible images of the patient's dentition. In some examples, the dentition state capture module 1020 can execute or perform any operations described with respect to the intraoral scanning system 910.

The alignment module 1030 can align elements or objects from the treatment plan with elements or objects from an image of the patient's dentition. For example, the alignment module 1030 can align teeth (teeth positions) that have been described by a treatment plan to teeth that have been captured through an image scan. In some examples, the alignment module 1030 may determine positions of the patient's upper jaw and lower jaw with respect to a temporomandibular joint (as described with respect to FIGS. 2-4) to determine teeth alignment.

The treatment recommendation module 1040 can determine whether any changes in the treatment plan may be needed to achieve a particular final position of teeth. For example, any misalignment between the position of teeth in an image scan and the position of teeth (such as a misalignment greater than a predetermined amount) described by the treatment plan may indicate an undesirable outcome. The treatment recommendation module 1040 can recommend a change in the treatment plan to address any potential undesirable outcome. On the other hand, if there is a misalignment less than a predetermined amount, then the treatment recommendation module 1040 may not suggest any changes in the treatment plan.

This invention provides a framework for jointly simulating a patient's upper and lower jaws, enabling a more comprehensive view of a patient's entire dentition (compared to a per-jaw basis approach) for virtual oral diagnostics and visualizations throughout an orthodontic treatment process.

As described herein, a system, apparatus, and/or method is described to provide a fully parameterized jaw pair modeling framework for simulating the relative poses/movements of a patient's upper and lower jaws (including individual tooth movements). This can allow flexible renderings of given jaws: panoramic, orthographic, perspective, etc. Also provided is the ability to conduct 3D dentition to 2D image alignment and infer per-tooth/jaw poses from multi-view patient photos. In some cases, utilizing the 3D to 2D alignment, this may improve tooth numbering predictions resulting directly from an ML model.

Some advantages include utilizing the 3D to 2D alignment, potentially allowing improved tooth numbering predictions resulting directly from an ML model. This may be extended to include morphable 3D gingiva/tooth models, instead of rigid 3D gingiva/tooth objects from 3D scans. The optimization of 3D morphable gingiva/tooth models could be particularly useful in restorative cases. These methods may not require GPU, though GPU acceleration is supported. In some examples, by conducting a 3D to 2D registration process a patient's 3D dentition may be aligned with his/her photos (open/closed bites from different views, e.g. anterior/right/left/occlusal). In other words, starting from a patient's initial dental scan (if available, otherwise, a generic morphable dentition may be used) showing the patient's actual dentition at the time of photo taking, one or more orthodontic assessment analyses may be easily and quickly performed, including (but not limited to) overbite, overjet, spacing, crowding etc. These methods and apparatuses performing them may take imperfect tooth segmentation/numbering results, and achieve reasonable 3D-to-2D alignment accuracy, providing a possibility of refining tooth segmentation/numbering in a post-processing step. In addition, these methods and apparatuses may also allow flexible dental renderings, that may be particularly useful for visualization and patient education purposes in dental practices, forward rendering to track the mapping between 3D mesh faces and corresponding 2D image pixels, for 2D-to-3D labeling, and to generate synthetic data from the 2D renderings of simulated jaw pairs (potentially even with GAN models) to allow more possibilities in various ML model training/improvements.

In general, these methods and apparatuses may perform relatively quick and easy set-up. For example, the jaw pair simulation framework may include individual tooth models (in the form of triangular meshes, or some parametric forms). Each individual tooth can have rotational and/or translational movements within the corresponding jaw. In addition, tooth models can also have morphable shapes. The jaw pair simulation framework (e.g., the 3D model) may include gingiva mesh objects (in the form of triangular meshes, also could potentially have morphable surfaces). In some examples, the upper jaw may consist of an upper gingiva object and a set of upper teeth. The upper jaw may be fixed in space, just as it is fixed to a patient's skull. The lower jaw may be consisted of a lower gingiva object and a set of lower teeth. One can parameterize the lower jaw movements in different ways with full 6 (or less) degrees-of-freedom. As an example, these methods may simplify the lower jaw movements as the combination of: rotation about the temporomandibular joint, and/or slight translational movements.

These apparatuses may be particularly useful for applications including 3D-to-2D registration for both jaws, allowing oral diagnostic measurements directly from 3D models. By simulating the upper and lower jaws jointly, and conducting a 3D-to-2D registration process to align the 3D jaw pair model to 2D photo(s), the underlying 3D jaw pair model of a patient may be determined, which may enable the performance of oral diagnostics directly on the obtained 3D model.

Alternatively or additionally, these methods and apparatuses may improve tooth numbering based on 3D-to-2D alignment. It may be generally difficult to train a pure image-based ML model for tooth numbering tasks, especially in cases with more complicated dentitions (missing teeth, erupting teeth etc.). Thus, the methods described herein may improving ML-tooth numbering models by utilizing the 3D dentition information from the treatment plan. FIG. 7A above shows an example in which a pure ML tooth numbering model mistakenly numbered tooth-25 as tooth-26. To fix this incorrect numbering, the patient's 3D dentition may be taken from the treatment plan, and the 3D dentition may be aligned to the 2D image (see, e.g., FIG. 7B).

In FIG. 7B the limited image quality and visibility of multiple teeth may result in relatively poor alignment, it is sufficient to correctly assign tooth-25 (because the “tooth-26” tooth blob in FIG. 7B mostly overlaps with the actual tooth-25 in treatment plan.) As a result, we can update the original tooth numbering in FIG. 7A and achieve the correct numbering as shown in FIG. 7C.

These methods and apparatuses may be useful for data synthesis using the jaw pair models described herein. For example, given a jaw pair, and the comprehensive jaw movement mechanism (TMJ properties) as described above, complete control of the 3D jaw model may be provided, and the bite opening and closing may be modeled at various degrees. As a result, tooth mask images (similar to FIGS. 5B and 6B above) may be generated from different viewpoints, at different degree of jaw openness. Furthermore, one or more generative adversarial networks (GANs) may be used to generate photo-realistic oral images, which could be useful to train various ML models for oral diagnostics, especially when real world high quality images are limited.

An of the methods and apparatuses described herein may be particularly useful for assisting in photo-based orthodontic assessment and/or diagnosis. For example, these methods and apparatuses may calibrate a 3D model of the patient's dentition to one or more two dimensional images that may be taken later, as compared to the initial scans (e.g., intraoral scans) used to generate the digital 3D models. Two-dimensional (2D) images may be used to calibrate the upper and lower jaw relationship between for the 3D digital model, and the same of or different 2D images, including images taken later, may be compared to the 3D digital model to provide helpful diagnostic and/or treatment, including treatment monitoring, information.

### Tooth Numbering

For example, the methods and apparatuses described herein may be used to determine and/or confirm tooth numbering. Tooth numbering may be an important steps towards successful photo-based orthodontic assessment and diagnostics. While existing AI/ML models can provide acceptable predictions on a good number of photos, for both tooth instance segmentations and numbering, there are cases where tooth numbering is particularly challenging for AI/ML models, due to the lack of knowledge of a patient's 3D dentition.

The joint jaw pair registration framework described herein provides an example of an approach to register a patient's upper and lower jaw teeth onto a patient's photo. Starting from an initial tooth segmentation and numbering mask (which may be inaccurate), the registration process described herein may find reasonable estimates for both camera parameters and the relationship between the patient's upper and lower jaw teeth, such that the projection of the patient's upper and lower jaws, under the estimated camera parameters, will align closely with what is in the patient's photo. The alignment between the projection of 3D model of the patient's teeth and a photo of the patient's teeth may be a bit off, depending on the accuracy of the initial tooth segmentation/numbering. As a result, the initial tooth numbering may be compared with the projected numbering from 3D teeth. For example, a Bayesian tooth numbering framework may be used, and may be used to correct numbering mistakes in the initial tooth segmentation/numbering mask, if any.

Compared with the single-jaw registration processes, which are largely limited to the analysis of just open bite photos; the joint jaw pair registration process described herein can be extended to closed bite photos, thanks to the inclusion of a lower jaw articulator model (allowing the estimate of the relationship between a patient's upper and lower jaws). For example, the registered 3D model (including parameters such as the TMJ parameters defining the movement and/or degrees of freedom as determined above) may provide joint jaw pair registration having additional cross-jaw references, including the relation between upper and lower teeth, for tooth numbering. In contrast, single jaw approaches may only provide tooth relations within the upper/lower jaw, respectively.

### Bite Class

The methods and apparatuses described herein may be particularly helpful for examining the bit relationship between the upper and lower jaws, once the jaws have been registered using the methods and apparatuses described herein. For example, an important application of the joint jaw pair optimization described herein may be the use of photographs (e.g., 2D images taken by the user and/or a dental professional) during, before or after treatment to provide dental occlusion monitoring. There are different types of dental occlusions, including class I, II, III malocclusion, crossbite, deep bite, open bite etc. To accurately determine the type of bite class and measure the degree of malocclusion from photos, the 3D model must be accurately estimated from 2D photos in high precision, and the relative position of upper and lower jaws may therefore be of particular importance. Under one unified camera system, it is easy to measure the distance of reference points on the registered 3D model including the upper and lower jaws and their relative possible movements (e.g., based on the parameters, such as TMJ parameters) to determine a consistent estimate of the pixel sizes for the input photo if the measurement is on 2D photos.

If separate jaw 3D-to-2D registration is used (e.g., without registering the upper and lower jaws of the 3D model and determining the parameters such as TMJ parameters, as described herein), the results may be much less accurate. For example, converting the lower jaw camera system to the upper jaw camera system can give a unified camera system, as the 3D-to-2D registration does not have unique solutions. However since this technique typically ignores the mechanics that connect the two jaws, it has extra freedom of how the jaws position relative to each other, and therefore the combined system typically will not reflect the natural setting of the jaws.

Bite class may be determined from the 3D digital model (that has been registered to the 2D image(s) as described herein) by measuring the distance between one or more teeth on the upper jaw as compared to one or more teeth on the lower jaw when the jaws are modeled in a particular position, such as when the jaws are closed. For example, in some cases bite class measurements may be made by taking the position of the maxillary canine relative to the mandibular teeth. The input may be a closed bite photo, generally in which most of the lower teeth are obscured by the upper teeth, making it difficult or impossible to do lower jaw registration alone. However with the use of joint pair optimization as described herein, in which the digital 3D model of the upper and lower jaws are registered using 2D images, the digital model may include the accurate relative movement of the lower jaw relative to the upper jaw (e.g., using the estimated parameters, such as TMJ parameter(s)). In this case, the lower jaw is part of the optimization, and the visible and covered parts of the lower and upper jaws are naturally derived from the setting of the jaw pair. This results in a higher accuracy of estimation of the actual 3D model, hence the measurement of distance between reference teeth and determination of bite class.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method comprising:
generating a three-dimensional (3D) alignment model based on a patient's 3D dental scans, wherein the 3D alignment model includes segmentation data of the patient's upper jaw portion and lower jaw portion;
generating a two-dimensional (2D) alignment projection based on the 3D alignment model;
determining a 2D difference between a patient's 2D dental image and the 2D alignment projection; and
importing dental information from the patient's 3D dental scan to the patient's 2D dental image when the difference is less than a threshold.

2. The method of claim 1, wherein generating the 3D alignment model includes determining a position of the lower jaw portion with respect to the upper jaw portion.

3. The method of claim 2, wherein the position of the lower jaw portion is constrained by a location of a joint coupling the lower jaw portion to the upper jaw portion.

4. The method of claim 2, wherein the position of the lower jaw portion is determined, at least in part, by a temporomandibular joint disposed with respect to the upper jaw portion.

5. The method of claim 1, wherein generating the 3D alignment model includes moving the lower jaw portion with respect to the upper jaw portion.

6. The method of claim 5, wherein moving the lower jaw portion is based on a location of a joint coupling the lower jaw portion to the upper jaw portion.

7. The method of claim 1, wherein generating the 2D alignment projection includes determining a location of the 3D alignment model and a location of a projection plane in a common virtual 3D space.

8. The method of claim 7, further comprising: projecting dental elements from the 3D alignment model to the projection plane, wherein the 2D alignment projection is based on the projected dental elements.

9. The method of claim 7, wherein the location of the 3D alignment model and the location of the projection plane is based, at least in part, on a point-of-view associated with a virtual camera disposed in the common virtual 3D space.

10. The method of claim 7, wherein a portion of the projection plane is cropped prior to determining 2D differences between the patient's 2D dental image and the 2D alignment projection.

11. The method of claim 7, wherein determining the location of the 3D alignment model includes locating the patient's upper jaw portion in 3D space using six degrees of freedom and locating the patient's lower jaw portion in the 3D space using one degree of freedom.

12. The method of claim 7, wherein generating the 2D alignment projection includes iteratively determining a position of the projection plane based on the difference between the patient's 2D dental image and the 2D alignment projection.

13. The method of claim 1, wherein the 2D dental image is based on a photo of the patient's dentition.

14. The method of claim 1, wherein the 2D dental image includes 2D information of an upper jaw portion and a lower jaw portion.

15. The method of claim 1, wherein determining the 2D differences between the patient's 2D dental image and the 2D alignment projection includes determining a difference between corresponding features of the patient's 2D dental image and the 2D alignment projection.

16. The method of claim 1, wherein the determining the 2D differences between the patient's 2D dental image and the 2D alignment projection includes determining a difference between outlines of corresponding dental structures of the patient's 2D dental image and the 2D alignment projection.

17. The method of claim 1, wherein the determining the difference between the patient's 2D dental image and the 2D alignment projection includes determining a difference between tooth boundaries determined from the patient's 2D dental image and the 2D alignment projection.

18. The method of claim 1, wherein the segmentation data is generated with one or more machine learning engines and one or more 3D models.

19. The method of claim 1, wherein the dental information includes tooth number information.

20. The method of claim 1, wherein generating the 3D alignment model further comprises iteratively determining a position of the lower jaw portion with respect to the upper jaw portion based on the difference between the patient's 2D dental image and the 2D alignment projection.

21. The method of claim 1, wherein generating the 2D alignment projection includes iteratively determining a position of a projection plane based on the difference between the patient's 2D dental image and the 2D alignment projection.

22. The method of claim 1, further comprising: generating a 3D image based on the 2D alignment projection.

23. The method of claim 1, wherein the patient's 2D dental image is a closed-bite photo.

24. The method of claim 1, wherein the patient's 3D dental scans are associated with a previously determined treatment plan.

25. The method of claim 1, wherein importing dental information from the patient's 3D dental scan comprises determining a bite class from the 3D dental scan.

26. The method of claim 25, wherein determining the bite class from the 3D dental scan comprises identifying one or more of: class I malocclusion, class II malocclusion, class III malocclusion, crossbite, deep bite, and/or open bite.

27. The method of claim 1, further comprising measure a degree of malocclusion from the patient's 3D dental scan.

28. The method of claim 1, further comprising determining or correcting tooth numbering using the patient's 3D dental scan.

29. A method comprising:
generating or accessing a three-dimensional (3D) alignment model of a digital model of a patient's upper jaw portion and a digital model of the patient's lower jaw portion, wherein the digital model of the patient's upper jaw portion and the digital model of the patient's lower jaw portion are based on one or more intraoral scans, wherein the 3D alignment model includes a TMJ parameter;
generating a two-dimensional (2D) alignment projection image from the 3D alignment model;
determining a difference estimate between a 2D dental image of the patient's teeth and the 2D alignment projection;
iteratively adjusting the TMJ parameter based on the difference estimate and repeating the steps of generating the 2D alignment projection image and determining the difference estimate until the difference is less than a threshold or until the number of iterations exceeds a second threshold; and.
outputting the 3D alignment model including the TMJ parameter.

30. A system comprising:
one or more processors; and
a memory configured to store instructions that, when executed by the one or more processors, cause the system to:
generate a three-dimensional (3D) alignment model based on a patient's 3D dental scans, wherein the 3D alignment model includes segmentation data of the patient's upper and lower jaw;
generate a two-dimensional (2D) alignment projection based on the 3D alignment model;
determine a 2D difference between a patient's 2D dental image and the 2D alignment projection; and
import dental information from the patient's 3D dental scan to the patient's 2D dental image when the difference is less than a threshold.
